(19) United States
(12) Patent Application Publication
(10) Pub. No.: US 2005/0220787 A1
(43) Pub. Date: Oct. 6, 2005

(54) Naturally occuring IgM antibodies that bind to lymphocytes

(76) Inventor: Peter Isaac Lobo, Charlottesville, VA (US)

Correspondence Address: PETER I. LOBO, 348 Keywest Dr., Charlottesville, VA, 22911, US

(21) Appl. No.: 11/139,566

(22) Filed: May 31, 2005

Related U.S. Application Data
(63) Continuation-in-part of application No. 10/292,002, filed on Nov. 7, 2002.

(30) Foreign Application Priority Data
Dec. 1, 2004 (WO) PCT/IB04/03966

(51) Int. Cl. A61K 39/395

(52) U.S. Cl. 424/143.1

(57) ABSTRACT

In this invention, the inventor discloses that naturally occurring IgM anti-lymphocyte antibodies bind to chemokine and non-chemokine receptors on lymphocytes and other cells, and downmodulate certain receptors including CD4 and CD2 on T cells and CD80 and CD86 on macrophages. The inventor also discloses that such antibodies (i) inhibit HIV-1 and other viruses from infecting cells (ii) inhibits activation and proliferation of T lymphocytes (iii) inhibits cytokine and chemokine production (iv) inhibits inflammatory processes, and (v) enhances death of malignant cells. This art or invention is novel in that the antibodies described herein are “naturally occurring” i.e. develop in absence of deliberate immunization and secondly these antibodies are distinct from disease causing autoantibodies in that these naturally occurring antibodies are polyreactive with low binding affinity.

### BACKGROUND ART

1. Field of the Invention

The present invention relates generally to naturally occurring IgM anti-lymphocyte antibodies and, more particularly, to a method of inhibiting disease progression through use of these antibodies.

2. Discussion of the Background

Normal humans and animals have naturally occurring auto-antibodies (referred to as NAA), which are produced in the absence of deliberate immunization with the target antigen. NAA can also bind to non-self antigens, which have the same or similar antigenic specificity as the autoantigen. Some of these NAA can be detected at birth, but the full repertoire of NAA develops later in life, usually by early childhood. Prior art has clearly demonstrated that NAA are mostly polyreactive in that a single monoclonal NAA can recognize several closely similar self antigens, which possess a unique but distinct set of epitope specificities. The nature of this polyreactivity is best exemplified by rheumatoid factor, which is an IgM NAA that recognizes and binds to the Fc region of different self and non-self IgG including the different IgG subclasses, but does not bind to other glycoproteins or self nucleo-proteins. The antigen binding site of NAA are in general encoded by germline genes, which are subjected to no or minimal mutation and this characteristic is responsible for the polyreactivity of these antibodies. Conversely, genes encoding the antigen binding site of antibodies produced in response to a foreign antigen or autoantibodies that cause disease (e.g. thyroiditis) are hypermutated and this genetic characteristic renders these antibodies highly specific with high binding affinity. Hence, the polyreactivity and low binding affinity of NAA resulting from their genetic makeup distinguishes these antibodies from the conventional antibodies produced after deliberate immunization or disease producing autoantibodies. Prior art teaches that NAA are predominantly of the IgM isotype but NAA of other isotypes have also been described (see Nakamura M, J of Immunol. 1988, vol 141, p 4165-72 and the material in this reference is incorporated herein by reference). Prior art has used antibodies, typically produced after immunization, and with high binding affinity and with high specificity, to protect against infections or to inhibit immune mediated disorders. The current invention is novel in that the antibodies used are naturally occurring. More information on NAA are reviewed in Lacroix-Desmazes S. et.al, J of Immunological Methods 216:117-137, 1998 and Cervenak J, Acta Microbiologica et Immunologica Hungaria 46:53-62, 1999 and the material in these two references is incorporated herein by reference.

Normal human and animals have in their blood low levels of circulating naturally occurring IgM antibodies that bind to their own leukocytes such as, for example, B and T lymphocytes, without causing cell lysis at 37° C. Such IgM antibodies are, therefore, referred to as “IgM anti-lymphocyte autoantibodies.” These IgM anti-lymphocyte antibodies bind to macrophages, neutrophils, endothelial cells and malignant cells and furthermore bind to allogenic cells in addition to autologous leukocytes. Both, animal IgM anti-lymphocyte NAA (mouse, rat, goat, horse, rabbit) and human IgM anti-lymphocyte NAA bind to the same human cells. Hence, in this application, IgM anti-lymphocyte auto-antibodies (whether human or animal) will be referred to as IgM anti-lymphocyte or leucocyte antibodies or autoantibodies, i.e. autoantibodies or antibodies will be used interchangeably. Prior art also teaches us that naturally occurring antilymphocyte antibodies are heterogenous comprising several different clones of IgM, each with a different speficity, but like other NAA's, each of these IgM clones can be polyreactive and therefore can bind to the same or similar, class of receptors. For example, prior art has shown that IgM Rheumatoid factor, like other NAA are polyreactive and will therefore bind to self and non-self IgG as well as all subclasses of IgG. The inventor shows that a monoclonal IgM isolated in his laboratory e.g., CK15 binds to CCR5, CCR3 and CCR1 and thus IgM anti-chemokine receptor NAA, like IgM Rheumatoid factor, can bind to different classes of chemokine receptors.

Levels of such anti-leukocyte antibodies increase during inflammatory states, including autoimmune diseases and infectious diseases such as, for example, systemic lupus erythematosus (“SLE”), sarcoidosis, HIV-1, malaria, Epstein-Barr virus (“EBV”) and cytomegalovirus (“CMV”). Individuals with asymptomatic HIV-1, therefore, have high levels of IgM anti-leukocyte autoantibodies.

The inventor's studies show, however, that chemokine receptors are one of the cell membrane receptors that bind to these IgM autoantibodies. The inventor shows that IgM inhibits binding of chemokines to their receptors, inhibits chemokine induced internalization of the chemokine receptor, and inhibits chemotaxis of normal leucocytes and malignant cells and through these mechanisms, the inventor believes that naturally occurring IgM anti-leucocyte antibodies inhibit the inflammatory processes and spread of malignant cells. The inventor's studies also show that IgM autoantibodies that bind to lymphocyte receptors are heterogeneous and show that IgM binds to the CD3 and CD4 receptor on T cells and in addition, downregulates CD2 and CD4 on T cells and CD80 and CD86 on macrophages. Accordingly the inventor shows that IgM NAA, by binding to CD3 and CD4 and by down regulating CD2, CD80 and CD86 inhibits T cell activation, cytokine production e.g., IL-13 and TNF-α and proliferation and also inhibits binding of HIV-1 to the CD4 receptor. The art teaches that T cell activation is important to initiate and maintain inflammatory process, and to upregulate membrane receptors. The art also teaches that T cell activation enhances entry and replication of different viruses including that of HIV-1 entry and replication (see Jenkins M K, Annual Review of Immunol, 2001, vol 19, p 23-45 and Huber B T, Microbiological Reviews, 1996 vol 60 p 473-82 for EBV, CMV Rabies virus; Sutkowski N, Immunity, 2001, vol 15, p 579-89 for EBV; Frenkel N, J of Virol, 1990, vol 64 p 4598-602 for Herpes Virus 6; Stein B S, Advances in Exp Med and Biol, 1991, vol 300 p 71-86 and Deeks S, Journal of Clinical Invest, 2004, vol 113, p 808-810 for HIV-1). All the material in these 6 references are incorporated herein by reference. Accordingly IgM NAA by inhibiting T cell activation has an inhibitory effect on inflammatory processes in different disease states and at different tissue sites as well as has an inhibitory effect on replication of HIV-1 virus and other viruses which are dependent on activation of T cells and other cells for viral replication.

The art also teaches that HIV-1 virus attaches to the CD4 receptor and enters cells through binding of the virus to chemokine receptors (e.g. CXCR4 and CCR5), which internalizes after viral binding. The art also teaches that replication of HIV-1 within the cell is enhanced with cell activation. Hence the inventor believes that IgM anti-leucocyte antibodies inhibit HIV-1 infection (i) by inhibiting HIV-1 virus binding to CD4 and chemokine receptors, (ii) inhibiting HIV-1 induced internalization of chemokine receptor and (iii) by inhibiting T cell activation, thus inhibiting viral replication.

The art also teaches that certain viruses bind to non-chemokine receptors on lymphocytes. Polio virus binds to CD155 receptor, Herpes virus 6 binds to a T lymphocyte receptor that has not been identified while the EBV virus binds to the CD21 receptor on B lymphocytes (See Dimitrov D S, Human Immunovirology, vol 2 p 109-121, 2004 for polio and other viruses; Barel M, Eur J of Immunol, vol 33, p 2557-2566, 2003 for EBV virus; and Frenkel N, J of Virol, vol 64, p 4598-4602, 1990 for Herpesvirus 6). The art also teaches that replication of these viruses is enhanced with activation of these cells. Hence the inventor believes that these heterogenous and polyreactive IgM anti-leucocyte antibodies will inhibit infectivity of these viruses by binding to non-chemokine receptors involved in viral entry and cell activation.

The art also teaches that many inflammatory processes are initiated by T cell activation, with enhancement of chemokine and cytokine production, and chemotaxis of cells. Accordingly, the inventor believes that IgM NAA inhibits inflammatory processes, by inhibiting activation, and proliferation of T cells and other cells, inhibiting chemokine and cytokine production, and by inhibiting chemotaxis of inflammatory cells.

The inventor will now briefly provide a summary of chemokines and chemokine receptors. Details on this subject are described by Olson and Ley, Amer. J Physiol Regulatory Integrative Comp Physiology 283: R7-R28, 2002; by Gerard and Rollins, Nature Immunol 2: 108-115, 2001; and by Onuffer and Horuk, Trends in Pharmacological Sciences 23: 459-467, 2002 and the material in these 3 references is incorporated herein by reference.

The known chemokine system in humans comprises, approximately 50 different chemokines and about 20 G-protein coupled chemokine receptors. The chemokine system has several characteristics (i) Most chemokines are secreted but some e.g. fractalkine are expressed on the cell surface. (ii) Chemokines are subdivided into CC, CXC, or CX₃C groups based on the number of amino acids between the first two cysteines (iii) Certain chemokines bind only one receptor e.g. CXCR4 with SDF-1α and CXCR5 with BCA-1 while other receptors can bind to several chemokines e.g. CXCR3 binds to IP-10, Mig and I-TAC. Similarly, a single chemokine can bind to several receptors e.g. RANTES will bind to CCR1, CCR3 and CCR5 with high affinity. This has led many in the field to suggest that the chemokine system was rife with redundancy. However, there are certain exceptions as lack of CXCR4 receptor expression is associated with abnormal embryogenesis and organogenesis. In addition, different chemokine receptors expressed on the same cell can induce specific signals, thus indicating that receptors are coupled to distinct intracellular pathways. (iv) Certain chemokines (and their respective receptors), important for normal homeostatic trafficking (e.g. BCA-1, which is involved with normal migration of lymphocytes to lymph nodes), are constitutively expressed while inflammatory chemokines (and their receptors) are induced on leucocytes and other cells e.g. endothelial cells, only under specific conditions, typically by inflammatory chemokines e.g. IL-1 or TNF-α produced by macrophages or activated T lymphocytes. (v.) Chemokine receptors are expressed on many different cells including leucocytes, endothelial cells, smooth muscle cells, and epithelial cells and neuronal cells and these cells can also secrete chemokines.

Chemokines play a prominent role in leucocyte trafficking that occurs with several inflammatory processes as diverse as multiple sclerosis, rheumatoid arthritis, systemic lupus erythematosus, vasculitis, allograft and xenograft rejections, acute and chronic bacterial and viral infections, asthma, colitis, psoriasis, atherosclerosis, hypertension, ischaemia-reperfusion and inflammation associated with neoplasia. Additionally, chemokines play a role in other non-inflammatory processes e.g. organo-genesis, hematopoiesis, and neuronal communication with microglia and with angiogenesis. The pivotal role played by chemokines in some of these disorders is illustrated by the observation that (a) specific deficiency of CXCR4 is associated with abnormal organo-genesis and (b) individuals with a homozygous defect in CCR5 are protected from allograft rejections and asthma. The participation of the chemokine system in inflammatory processes involves leucocyte trafficking as well as leucocyte activation and immune cell differentiation. For example, chemokines induce neutrophils to increase integrin expression, neutrophil degranulation and super oxide formation. Similarly, the chemokine system is involved in tissue-specific homing of lymphocyte subsets to lymphoid organs where lymphocytes get activated and start differentiating (see Olson and Ley reference).

Of particular significance is the finding that chemokine receptors i.e. predominantly CXCR4 and CCR5 act as co-receptors for the entry of HIV-1 virus into cells. The X4 HIV-1 virus uses the CXCR4 receptor while the R5 HIV-1 virus uses the CCR5 receptor. It has become abundantly clear that viral entry through chemokine receptors is of prime importance in influencing viral replication and disease progression after an HIV-1 infection. For example, individuals with genetic defects in the CCR5 receptor have been associated with a prolonged latency period after HIV-1 infection i.e. a slower progression of HIV-1 to AIDS.

Researchers and pharmaceutical companies have been looking into strategies to block or inactivate specific chemokine receptors in an effort to inhibit inflammatory processes that induce disease processes and to inhibit HIV-1 entry into cells. Some of these include use of peptides and IgG monoclonal antibodies that will bind to specific chemokine receptors. Such strategies, however, have not as yet been shown to be effective.

Finally, the inventor will provide a summary on the role of T cells in inflammatory processes. Prior art has shown that T cells play a prominent role in several diverse inflammatory processes including allergy, autoimmune disorders, rejection of transplant organs, atherosclerosis, and resistance to infections. For example, allografts are not rejected in T cell deficient animals indicating that T cell activation and cytokine production is necessary to induce or facilitate the inflammatory process associated with rejection. The art also teaches that CD3, CD4, and CD86 are important receptors (or switches) that are involved in T cell activation (see Werlen G, Current Opinion in Immunol Vol 14 p 299-305, 2002 for prior art in this regard). The inventor therefore believes that IgM NAA by binding to CD3 and CD4 will inhibit T cell activation and provide another mechanism to inhibit diverse inflammatory processes where T cells activation plays a prominent role. Examples on the role of T cells in some inflammatory processes are reviewed in Perkins D L, Current Opinion in Nephrology and Hypertension Vol 7, p 297-303, 1998; Hansson G K et al Circulation Research Vol 91 p 281-291, 2002 and the material in these two references are incorporated herein by reference. There is prior art to show that cytokines an chemokines are involved in the inflammatory process. Certain cytokines and chemokines are pro-inflammatory while others are anti-inflammatory. Prior art has shown that TNF-α in particular, is the major cytokine that enhances inflammation in rheumatoid arthritis, psorasis and Crohn's disease. Inhibitors of TNF-α have a marked beneficial effect on these particular inflammatory disorders (see Feldman M, Annual Rev Immunol 2001, vol 19, p 163-196; Sandbom W J, Inflamm Bowel Dis 1999, vol 5, p 119-133; and Chaudhari U, Lancet 2001, vol 357 p 1842-1847). In the present application, inventor has demonstrated that anti-lymphocyte NAA inhibits leucocyte secretion of TNF-α and other chemokines. Inventor believes that inhibition of chemokines and cytokines by anti-lymphocyte NAA could provide another mechanism for inhibiting an inflammatory process.

Researchers and pharmaceutical companies have been looking into strategies to inhibit T cell activation, chemokines, cytokines, and chemotaxis in an effort to inhibit inflammatory processes including autoimmune disorders, allergies and allograft rejections. Some of these include use of antibodies that inactivate or kill T cells. These antibodies are produced by immunizing animals with human T lymphocytes. Other strategies include use of (i) immunosuppressive drugs e.g. cyclosporine or Rapamycin and (ii) agents that inhibit cytokines produced by activated T cells. Such strategies are expensive and have serious side effects and have to be taken for prolonged periods and at times for life especially after a transplant. Vaccines that can enhance production of IgM NAA may prove to be much less expensive, more effective and available for large populations of individuals.

### SUMMARY OF THE INVENTION

Normal humans and animals have naturally occurring IgM autoantibodies (referred to as IgM NAA), some of which are present at birth and the full repertoire of these antibodies develop in the first few years of life. These antibodies are produced in the absence of deliberate immunization with the target antigen. IgM NAA are distinct from antibodies produced after immunization with foreign antigen or from autoantibodies that cause disease, in that the antigen binding site of NAA are encoded by germ line genes, which undergo minimal or no mutation. As a result, IgM NAA are polyreactive and have low binding affinity. IgM NAA are mostly polyreactive in that a single IgM monoclonal antibody can recognize several closely similar self-antigens, which possess a unique but distinct set of epitope specificities. Hence, a monoclonal IgM that binds to one receptor will very often bind to similar receptors belonging to the same class, e.g. an IgM antibody to CCR5 could bind to another chemokine receptor e.g. CCR1. While the presence of IgM anti-lymphocyte NAA has previously been described, there is no prior art identifying the glycoprotein lymphocyte receptors targeted by IgM, nor is there prior art showing that IgM anti-lymphocyte NAA can inhibit T cell function or inhibit viral infectivity of cells, or inhibit cytokine production or inhibit chemotaxis.

In the present invention, applicant has discovered that some of the IgM anti-lymphocyte NAA obtained from normal human sera bind to chemokine receptors and specifically inhibit binding of chemokines to their receptors, inhibit chemotaxis and inhibit HIV-1 from infecting cells. The inventor has also shown that IgM NAA inhibit T cell activation, inhibit cytokine production and inhibit T cell proliferation. Accordingly, the inventor believes that IgM NAA inhibits HIV-1 infectivity by “blocking” HIV-1 entry through binding to CD4 and the chemokine receptor as well as by inhibiting lymphocyte activation.

Moreover, IgM anti-lymphocyte NAA are a heterogenous group of several different antibodies that bind to chemokine and other non-chemokine receptors on the lymphocyte. Such non-chemokine receptors include glycoprotein and glycolipid receptors. These IgM anti-lymphocyte NAA have low binding affinity and do not lyse normal cells in the presence of complement at body temperature (i.e. 37° C.). Applicant, in this invention has discovered that these polyreactive IgM anti-lymphocyte NAA bind to the same or closely similar lymphocyte receptors that are also present on other leucocytes (i.e. neutrophils, eosinophils, and macrophages), endothelial cells and malignant cells (both lymphoid and non-lymphoid). In the present invention, applicant also demonstrates that IgM anti-lymphocyte NAA binds to a non-chemokine receptor, identified as CD3 and CD4 and further shows that naturally occurring IgM with anti-CD3, anti-CD4 and anti-chemokine receptor activity inhibits lymphocyte activation and proliferation. Applicant also demonstrates that IgM antilymphocyte NAA downregulates CD2 and CD4 on T cells and CD80, CD86 on macrophages, (which are antigen presenting cells) thus inhibiting T cell activation through this additional mechanism.

The inventor has observed that human kidney transplant recipients, who have high levels of IgM reactive to their donor lymphocytes rarely, have problems with rejections. Applicant, in this invention, believes that protection against rejection is mediated by the inhibitory effect of IgM on autologous leucocytes and donor endothelial cells. High level binding of recipient IgM to donor lymphocytes is also associated with similar level of IgM binding to recipient leucocytes and donor endothelial cells. Recipient IgM would thus protect against rejection by inhibiting leucocyte chemotaxis as well as by inhibiting activation of autologous lymphocytes e.g. through binding to CD3 and CD4 and chemokine receptors as well as by inhibiting chemokine and cytokine production and/or activity.

Finally, the inventor has observed increased apoptosis of malignant cells, (but not normal cells) at 37° C. in presence of normal IgM anti-lymphocyte NAA. The inventor believes that these antibodies also protect against malignancy by enhancing apoptosis and also by inhibiting metastatic spread of malignant cells, mediated through chemokine receptors. There is prior art to show that metastatic spread of malignant cells is enhanced by chemokine receptors.

Accordingly, one object of the present invention is to provide a method of inhibiting disease processes involving. and/or mediated by chemokine and non-chemokine receptors through use of IgM anti-lymphocyte NAA.

The above and other objects, advantages and features of the present invention will become more apparent from the following detailed description of the presently preferred embodiments, when considered in conjunction with the figures, and to the appended claims.

### DISCLOSURE OF INVENTION

To achieve the foregoing and other objects, and in accordance with the purpose of the present invention as embodied and broadly described herein, the present invention relates to the expression, stimulation and administration of isolated IgM antibodies to an individual to address viral infections and disease states.

Prior art has shown that IgM autoantibodies present in the blood of normal uninfected individuals and in newborns bind to extracellular receptors present on lymphocytes. There is prior art to also show that IgM autoantibodies to lymphocytes, which are present at low levels in normals, increase in various infectious states (including HIV), autoimmune disorders, and inflammatory disorders. These IgM antibodies are heterogenous and bind to several different membrane receptors including glycosphingolipid and phospholipid membrane antigens on the lymphocyte membrane. These IgM autoantibodies do not damage normal cells at 37° C. as at that temperature they have a low binding affinity and cannot activate complement.

According to the present invention, IgM anti-lymphocyte auto antibodies present in normal sera bind to chemokine receptors, for example, CXCR4, CCR5, CCR3 and CCR2b and other non-chemokine lymphocyte-surface receptors e.g. the CD3 and CD4 antigen. The inventor also shows that IgM anti-lymphocyte antibody inhibits HIV-1 from infecting cells.

While not wishing to be bound to a specific theory, the inventor believes that the increase in these antibodies after an HIV-1 infection, slows down the progression of the infection towards development of AIDS and the high levels of these IgM antibodies in newborns protect newborns from getting HIV-1 viremia from their infected mothers. Only 20 to 25% of babies, born of untreated mothers infected with HIV-1, are found to have the HIV-1 virus. Mechanisms for inhibiting HIV-1 infectivity of cells include, (but are not limited to): (i) inhibiting binding of HIV-1 to the CD4 receptor (ii) “blocking” of HIV-1 viral entry through binding of IgM to chemokine receptors (iii) inactivation of lymphocytes by binding to the CD3 and CD4 receptor or downregulating other activating receptors e.g. CD2, CD4, CD80, CD86 and chemokine receptors and inhibiting internalization of chemokine receptors after HIV-1 binds to these receptors. Lipid rafts contain glycosphingolipids as well as phospholipids, which prior art has shown to be target antigens for IgM anti-lymphocyte autoantibodies. The binding of IgM anti-lymphocyte NAA to glycolipids and phospholipids has been described in Griggi et al, Scand. J of Immunol, 40: 77-82, 1994 and Stimmler et al, Archives of Internal Medicine 149: 1833-1835, 1989 and this material is incorporated herein by reference.

Chemokines, chemokine receptors, and other lymphocyte receptors (e.g. CD3, CD4 and other co-stimulatory molecules) are involved in inflammatory processes that involve leucocytes and endothelial cells. Examples of inflammatory processes include (but are not limited) auto-immune disorders (e.g. SLE, rheumatoid arthritis), asthma, atherogenesis, end-stage renal disease (ESRD) patients on hemodialysis, sarcoidosis, various viral, bacterial and parasitic infections, allograft and xenograft rejections, various forms of vasculitis, multiple sclerosis, interstitial lung and kidney inflammation and glomerulonephritis. While not being bound to a specific theory, the inventor believes that IgM anti-lymphocyte NAA through binding to chemokine receptors and other lymphocyte receptors could inhibit the above-mentioned inflammatory processes. Potential mechanisms for inhibition would include inhibition of chemokine receptor function after binding of IgM and more importantly inactivation of lymphocytes and/or macrophages after binding to chemokine receptors and non-chemokine receptors as for example, the CD3, CD2 and CD4 and CD86 receptor. Another mechanism involves inhibition by IgM NAA, of cytokine and chemokine secretion by cells.

IgM anti-lymphocyte NAA also bind to endothelial cells and malignant cell lines. In this invention we show that IgM NAA, are polyreactive and hence, bind to the same or closely similar receptors present on these cells. The inventor believes that some monoclonal IgM anti-chemokine receptor antibodies are polyreactive and bind to several different chemokine receptors as absorption of IgM with lymphocytes removes the IgM that binds to malignant cells, Neutrophils, eosinophils, macrophages, and endothelial cells even though these cells have different chemokine receptors and lack chemokine receptors present on lymphocytes.

It is believed that IgM by binding to chemokine and other non-chemokine receptors on endothelial and malignant cells inhibit the function of these cells. For example, there is prior art to show that chemokine receptors on malignant cells contribute to metastases of these cells (see Mueller A et al, Nature Vol 410 p 50-56, 2001 and Gerard C, Nature lnnunol Vol 2 p 108-115, 2001). The inventor therefore believes that IgM, by binding to chemokine receptors on malignant cells and/or endothelial cells could inhibit the growth and spread of malignant cells. Furthermore there is prior art to show that lymphocytes in lymph nodes and infiltrating leucocytes within the tumor mass secrete chemokines and other cytokines, all of which contribute to growth and metastases of tumor cells. The inventor therefore believes that IgM by binding to chemokine receptor and other “activation” receptors on leucocytes and malignant cells as well as by inhibiting production of chemokines and cytokines will, through these additional mechanisms, also inhibit tumor growth and metastases. Finally, the inventor shows that malignant lymphoma cells (but not normal cells) have enhanced cell death at 37° C. when incubated with IgM. Hence, IgM through enhancing cell death of malignant cells could provide yet another mechanism for an anti-cancer effect.

Endothelial cells and leucocytes are also important in several inflammatory processes (e.g. allograft rejections, atherogenesis, vasculitis and inflammatory states of the brain). It is therefore believed that IgM anti-lymphocyte NAA by binding to chemokine receptors on leucocytes and endothelial cells could provide a protective role in inhibiting such inflammatory processes. Furthermore, IgM anti-lymphocyte NAA could also inhibit inflammatory processes by inhibiting receptors (e.g. CD3 and CD4) that activate lymphocyte and macrophages as well as by inhibiting production of chemokines and cytokines.

The inventor believes that pooled IgM preparations contain a heterogenous group of antibodies that bind to chemokine or non-chemokine receptors on leucocytes, endothelial cells and malignant cells and that the binding of IgM to several of these receptors may add or have a synergistic effect in IgM mediated inhibition of viral infectivity, inflammatory states and malignant cell growth and spread.

Experimnental Studies

Methods/Procedures

Cell Lines

Sup T-1 and Jurkat are human lymphoma T cell lines constitutively expressing the CXCR4 receptors. U937 is a human monocytoid cell line expressing CD4, CXCR4, CCR5 and other chemokine receptors e.g., CCR 2b. HuT-78 is a human lymphoma T cell line constitutively expressing CXCR4 and CCR5. These cell lines are obtained from the AIDS Reagent Program or ATCC at NIH.

An HOS osteosarcoma cell line is co-transfected with CD4 and either CXCR4 or CCR5 or CCR3 or CCR1 genes to produce HOS-CD4, HOS-CD4-CXCR4 and HOS-CD4-CCR5 HOS-CD4-CCR3 and HOS-CD4-CCR1 cells. GHOST CCR5 and GHOST CXCR4 are HOS-CD4 cells co-transfected with the HIV-2 LTR driving hGFP construct and either CCR5 or CXCR4 genes, respectively. The cell line and the transfectants are obtained from the AIDS Reagent Program at NIH.

A glioblastoma cell line, U373-MAGI, is co-transfected with CD4 and either CXCR4 or CCR5 to produce U373-MAGI-CXCR4 and U373-MAGI-CCR5, respectively. Again, the cell line and the transfectants are obtained from the AIDS Reagent Program at NIH.

All of the transfected cell lines stably express CCR5 or CXCR4, with the U373-MAGI cells having the highest expression of these receptors.

Human peripheral blood lymphocytes (“PBL”) is activated with IL-2 to enhance CCR5 and CXCR4 expression. PBL (2×10⁶ cells in 1 ml RPMI culture media containing 10% fetal calf serum are activated by initially pre-treating Ficol/hypaque separated PBL with IL-2 (40 units/ml) and phytohemagglutinin (“PHA-P”, 5 mcg/ml) and then washing the PBL after the cells are cultured at 37° C. in about 5% CO₂ for 24 to 48 hours. Such PHA pre-treated cells are then kept growing for about another 6 to 7 days supplemented with 20% fetal calf serum and IL-2 (40 units/ml) before being used in chemokine binding assays.

HIV-1 Viruses

The R5 HIV-1 viruses (8397, 8442, and 8658) used to infect GHOST CCR5 or mitogen activated PBL are obtained from Dr. Homayoon Garadegan at Johns Hopkins University. The X4 virus IIIB and RF used to infect GHOST CXCR4 or mitogen activated PBL is obtained from the AIDS Reagent Program at NIH.

IgM Preparations and Sera

Studies were performed with IgM that was purified from heat-inactivated sera (56° C.) of normal individuals or from patients. IgM was prepared from sera with Sephacryl S-300 HR size exclusion column chromatography. IgM was not precipitated from sera with hypotonic dialysis or by ammonium chloride precipitation as these processes reduced the biological activity. Any contaminating IgG was removed from the IgM preparation by re-passage of purified IgM through a Sephacryl/S-300 HR column and by exposure to Agarose-protein G and Agarose bound to goat anti-human IgG (Sigma). We employed size column chromatography basically to remove low molecular weight substances (e.g. chemokines, anti-viral drugs) and IgG anti-HIV-1 antibodies that could affect our data. Serum protein electrophoresis and immunoelectrophoresis revealed that these IgM preparations, obtained by size exclusion chromatography, contained IgG (<1%), albumin (<3%), and other proteins (<1%). We did not want to affinity purify these antibodies as such procedures, e.g. binding of IgM to mannan binding protein or binding of IgM to agarose coupled with goat anti-human IgM yielded 10-15% of the starting IgM and has the potential of depleting certain IgM subsets. Instead, in several experiments we used IgG, IgA, albumin and alpha 2 macroglobulins to determine if our observations could be explained by some of these minute contaminants. No detectable RANTES and SDF-1α was present in these IgM preparations when analyzed by ELISA and Western blot techniques.

We obtained sera from normal uninfected healthy individuals, asymptomatic patients with HIV-1 infection, and on HAART therapy and patients with end stage renal disease (ESRD) on hemodialysis. Some of the HIV-1 patients had suffered AIDS defming illnesses and some other had high viral loads (>100,000 copies) despite HAART therapy. To obtain a sufficient quantity, IgM from nine HIV-1 patients was pooled. IgM from seven ESRD patients was also pooled. Data presented in figures are either from individual or from pooled IgM and are indicated in the figures.

The culture supernatants of EBV transformed human B cell clones are separated by Sephacryl S-300 HR column chromatography, which separates proteins by size. The human B cell clones are derived from B lymphocytes isolated from the blood of a patient with SLE. The B cell clones are developed by infecting B cells with the EBV virus, which makes the B cells immortal and capable of secreting a specific antibody, i.e., IgM. More particularly, non-T cells are isolated from PBL after removal of T cells using a sheep erythrocyte rosetting technique. About 2×10³ non-T cells in about 0.1 ml RPMI 1640 cell culture media containing about 10% fetal calf serum are added to each well of a 96 well plate. To each well is then added about 50 lambda of EBV-containing B95-8 cell line supernatant. Before incubation, about 10⁴ allogenic irradiated (3,000 rads) PBL in 0.05 ml are added as feeder cells. The plates are incubated at 37° C. in about 5% CO₂. The culture medium is replaced about every 4 to 5 days. After about 3 to 4 weeks, B cell lines appear as “clumps” in the wells. Feeder cells die during this period. When the “clumps” appear, these clumped cells are transferred to a 24-well plate, i.e., cells from one well are transferred into a single larger well. Culture media is changed when the media changes to a yellowish color, usually about 3 to 5 days. After about 2 weeks, supernatants are checked for IgM antibody. Wells containing lines with desired antibody specificity are further subcloned with limiting dilution in a 96-well plate. About 10⁵ feeder cells are added to each well containing these lines. Supernatants are rechecked to isolate clones with desired antibody specificity. Supernatants are refrigerated, but not frozen as IgM can precipitate out. Clones secreting IgM antibodies that are useful in inhibiting HIV-1 infectivity are cryopreserved. Supernatants from such clones usually contain about 0.5 to about 0.7 μg/ml antibody. Clones of particular interest can be fused with K6H6/B5 plasmacytoma cell line (or other similar cell lines that do not secrete antibodies) to develop hybridomas. The clones are screened to identify and obtain those clones that react with CD3, CD4, CCR5 and CXCR4 chemokine receptors present on the transfected cells. Such clones have increased IgM binding by flow cytometry to the HOS-CD4 transfectants (i.e., HOS-CD4-CXCR4 and HOS-CD4-CCR5) when compared to the HOS-CD4 control. Two clones, CK12 and CK15 secreting IgM with increased binding to HOS-CD4 CCR5 or CXCR4 transfectants were identified in this manner. CK12 only bound to HOS-CD4-CXCR4 while CK15 was polyreactive and bound to HOS-CD4-CCR5, HOS-CD4-CCR3 and HOS-CD4-CCR1.

Any contaminating IgG is removed from the IgM preparations that are isolated from the sera and the culture supernatants by exposure to both protein G-Agarose (available from Sigma) and goat anti-human IgG (Fc specific)-Agarose (available from Sigma).

IgM is also obtained using Sephacryl S-300 HR column chromoatography from sera of a patient diagnosed with Waldenstrom macroglobulinemia (a form of B cell lymphoma) and which, on serum protein electrophoresis, has a single peak for IgM (monoclonal). This latter IgM preparation is hereinafter referred to as “Waldenstrom IgM” and the monoclonal IgM binds to an undefined membrane receptor on lymphocytes and other leukocytes =p IgM was also obtained from pooled sera of mice, rats, goats and rabbits. We used similar techniques as for human sera to obtain purified animal IgM.

Absorption of IgM with Jurkat and U937 Cells

2.5 ml IgM at 0.2 mg/ml in RPM1 was absorbed for 45 minutes with 280×10⁶ Jurkat cells and 200×10⁶ U397 cells at 37° C. in 5% CO^{2.} We used Jurkat and U937 cells as these cells express most of the leucocyte membrane receptors including CD3, CD4 and chemokine receptors. The IgM was centrifuged at the end of 45 min to remove cells and the absorbed IgM was quantitated using ELISA techniques. 25 to 30 percent of IgM was lost in the absorption technique. Absorbed IgM had <5% residual binding activity to Jurkat cells, U937 cells, lymphocytes, neutrophils or cultured endothelial cells as determined by flow cytometry.

Preparation of Monomeric IgM

Monomeric IgM was made from the pentameric form in 200 nM Tris, 150 mM NaCl, and 1 mM EDTA, pH 8.0, and by reduction with 5 mM DTT for 2 hour at room temp. Subsequent alkalinization was performed for 1 hour on ice with 12 mM iodoacetamide. IgM monomers were isolated from any remaining pentameric forms by column chromatography (Superdex-200) equilibrated with PBS. Purity of monomeric IgM was confirmed with SDS-PAGE Western blots under reducing and non-reducing conditions. With flow cytometry, one observed less than 20 percent reduction in binding of monomeric IgM to lymphocytes when compared to the pentameric form.

Chemokines

RANTES, SDF-1α and biotin-labeled SDF-1α-MIP-1α and RANTES are obtained from Becton Dickinson of La Jolla, Calif. Radio-labeled RANTES (referred to as “I¹²⁵ RANTES” or “I¹²⁵”) is obtained from NEN Life Science of Boston, Mass. RANTES binds to CCR5, while SDF-1α binds to CXCR4.

Antibodies

Clones 2D7, CTC-5, 45502, 45523, and 45549 are murine IgG monoclonal antibodies specific for CCR5 when expressed on intact cells. Clone CTC-5 in addition binds to linearized CCR5 in Western blots. Clones 12G5 (IgG 2a) 44708, (IgG 2a) 44717 (IgG 2b), and 44716 (IgG 2b) are murine IgG monoclonals that bind to CXCR4 receptors on intact cells and neutralize chemotaxis in response to SDF-1α. All these antibodies were obtained from R&D Systems or the NIH AIDS Reagent program. Clone 4G10, a murine IgG monoclonal that binds to the N-terminal region of CXCR4 was a kind gift from Dr. Chris Broder. Leu 3a (Becton-Dickenson) is a murine IgG monoclonal specific for CD4.

IgM Inhibition of Chemokine Binding to Receptors on Intact Cells

Normal, ESRD and HIV IgM have a similar inhibitory effect on binding of biotin labeled SDF-1α and MIP-1α to cells. Cells (0.5×10⁶ in 0.5 mL) obtained from T cell lines (Hut 78 and Jurkat E-6) or Monocytoid cell line (U937) or PBL activated for 3 days with PHA+IL-2 were incubated with or without IgM (1 to 30 μg/1×10⁶ cells/ml) in PBS buffer containing CaCL₂ at 37° C. for 45 min, and without a wash step, cells were re-incubated at 37° C. for 45 min with biotin labeled cytokine (50 ng). Cells were then re-washed in the cold and stained with PE-streptavidin.

Immunoprecipitation Technique and Western Blot Procedure to Detect IgM Binding to Solubilized Cell Membrane Receptors

Cell lines (80×10⁶) were incubated for 30 min at 4° C. with 10 ml of 100 mM (NH4)₂S0_{4,}20 mM Tris HC1 (pH 7.5) containing 10% glycerol, 1% Cymal −5 (Anatrace, Maumee, OH) and 1 tab mini-complete (Roche) to solubilize membrane receptors with minimal denaturation. IgM/receptor complexes were formed by interacting 100 μl of cell lysate (containing the equivalent of 50×10⁶ cells) with 100 ,ug of IgM. The mixture of IgM/cell lysate was then interacted with 50 μl of washed Agarose bead pellets containing covalently bound goat IgG anti-human IgM (Sigma). The agarose bead with bound IgM/receptor complexes was washed x3 (700 rpm) with Tris buffer containing 1% bovine albumin and 0.01% Cymal-5 and x2 with buffer containing 0.01% Cymal-5 The washed beads with IgM/receptor complexes were then interacted with Laemmli buffer containing 4% 2-ME and incubated at 37° C. for 30 minutes to dissociate and linearise receptors under minimal reducing conditions. Incubating at higher temperatures led to dissociation and denaturation of the goat IgG (covalently bound to the Agarose bead). Supernatants were then electrophoresed in SDS-PAGE and transferred on to nitrocellulose and then probed with primary IgG antibodies specific for the receptor in question. It was not unusual for the secondary HRP conjugated antibody (even if specific for the primary mouse or rabbit Fc fragment of IgG) to bind to extra protein bands of both the heavy and light chains of goat IgG (that disassociated from the beads) as well as the light chains of IgM. Hence the secondary antibody was routinely pre-absorbed with goat IgG and human IgM prior to use. In some experiments, we resorted to using unlabeled secondary antibody specific for goat IgG (H & L), especially if the primary antibody was of non-goat origin. Additionally as negative controls, the Western blot procedure was performed with supernatant from beads that were interacted with IgM (but with no cell membrane lysate) or with beads that were interacted with lysate (but with no IgM) so as to identify presence of non-specific bands. As a positive control the membrane lysate without beads or IgM was interacted with Laennnli buffer under similar conditions and then electrophoresed in SDS-PAGE.

Antibodies for Western Blots

The following antibodies were used as primary antibodies in the Western blot procedure: Polyclonal IgG rabbit antibodies to IL2-R (α or β chain), CD3, CD4, HLA-A, HLA-DR, or CXCR4; monoclonal mouse IgG antibodies to CCR5 (clone CTC, N-terminal) and CXCR4 (clone 4G10 N-terminal). Antibodies were obtained either from R & D Systems, MN, or Santa Cruz Biotechnology, CA or Biochain Institute, CA. The following HRP conjugated secondary antibodies (Fc fragment specific) were used: polyclonal IgG goat antibodies to rabbit IgG, mouse IgG, or human IgM. All secondary antibodies were obtained from Jackson Immunological Labs.

Chemotaxis Assay

This assay was performed using the 24 well Costar transwell tissue culture inserts with 5 micron polycarbonate filters. 0.15×10⁶ cells in 0.15 ml RPMI with 0.5% human albumin were added to the upper transwell. Thirty minutes later 100 ng of SDF-1α or RANTES or MIP-1α were added to the bottom well containing 0.6 ml of the same media as in the upper well. The chemotaxis assay was performed at 37° C. for 2 hours for activated PBL, 4 hours for Jurkat cells and 12 hours for Hut78 cells. Cells migrating to the bottom well were enumerated by flow cytometry. Chemotaxis index was calculated by dividing the total number of cells migrating in presence of chemokine by the number of cells migrating in the absence of chemokine. As a control for chemotaxis, four-fold chemokine was added to the upper transwell in presence or absence of chemokine in the bottom well. The effect of IgM on chemotaxis was evaluated by incubating IgM (5 to 30 μg/ml) with cells at 37° C. for 30 min prior to adding cells to the upper transwell.

MLR Assay

Briefly, 0.15×10⁶ PBL in 0.15 ml RPM1 containing 10% fetal calf serum were co-cultured (in triplicate) in flat bottom wells with similar number of cells from another individual known to have different HLA-Class 1 and DR antigens. After 5 to 6 days in culture, [H]³ Thymidine was added to cells in each well of a 96 well plate and 12 to 18 hours later cells were harvested over a filter matrix and the uptake of Thymidine by proliferating cells was quantitated using a liquid scintillation counter. Different doses of IgM was added on Day 0 and Day 1 of the culture period.

Quantitation of Cytokines in Culture Supernatants

Cytokines in PBL culture supernatants were assayed in a semi-quantitative manner using the Ray Bio Human cytokine Array #3 kit (Ray Biotech, GA) which consists of a membrane array containing 42 different primary murine antibodies, each specific for a cytokine. One ml of supernatant is incubated for 2 hours with the membrane which is then washed and re-incubated for one hour with a cocktail of the same 42 primary antibodies. After re-washing, the membrane is incubated with an HRP conjugated secondary antibody. Cytokine positive spots are detected on an X-ray film and quantitated with a densitometer. Significant changes in cytokine levels as detected by the Ray Bio assay was confirmed and quantitated with an ELISA technique.

Quantitating Phosphorylation of Intra-cellular Zap-70

Studies on phosphorylation of Zap-70 were performed with freshly obtained PBL and phosphorylation was quantitated at 0, 2, 5 and 10 mins (early stage) or at 16 hrs (late stage). In these studies, cells (0.6×10⁶/0.6 ml) were initially incubated with or without IgM (final conc 5 to 15 μg/ml) for 30 to 45 min at 37° and were then activated with immobilized anti-CD3 (OKT3) (1 μg of antibody in a well of a 48 well plate). Cells were then incubated for the required time at 37° in RPMI media with HEPES buffer and no fetal calf serum (FCS) for the “early stage” experiments and in the same media with 5% FCS and in 5% CO₂ for the “late stage” experiments. Phosphorylation of Zap-70 was evaluated in the absence (to determine background phosphorylation) or presence of immobilized anti-CD3. PBL activated for the desired length of time were immediately chilled in ice for 10 mins prior to fixing and permeabilisation. Cells were then stained with antibodies for the phosphorylated signaling protein or for the total signaling proteins and antibody binding to the signaling protein was quantitated by flow cytometry.

Temperature Dependence for the Cytolytic Effects of IgM Anti-leukocyte Antibody

Temperature dependence for the cytolytic effects of IgM anti-leukocyte antibody is evaluated by a complement dependent microlymphocytotoxicity assay. Various dilutions of IgM antibody are reacted for 1 hour with either 2×10⁵ PBL or IL-2-activated PBL (7 days) before adding fresh rabbit serum as a source of complement. After about 2 hours, the cells are washed twice before adding trypan blue and enumerating dead cells that stain blue. Experiments are performed at 15° C. and 37° C.

IgM Inhibition of HIV-1 Infection of Cells

a) HIV-1 Infection of GHOST Cells
   It has been observed that the HIV-1 R5 virus utilizes CCR5 receptors for cell entry, while the HIV-1 X4 virus uses CXCR4 receptors. Studies are conducted, therefore, to determine whether IgM inhibits HIV-1 entry into cells in light of such observations. In these studies, GHOST CCR5 and GHOST CXCR4 transfectant cell lines are infected with HIV-1. The GHOST cells are derived from HOS cells transfected with either CCR5 or CXCR4 genes-and also co-transfected with the HIV-2 LTR driving hGFP construct. The hGFP construct enables cells infected with HIV-1 virus to emit a green fluorescence so that the number of infected cells can be quantified using flow cytometry. These cell lines are particularly suited for these studies because single-cycle viral replication can be detected in less than 48 hours.
   About 2×10⁴ each of GHOST CCR5 and CXCR4 cells are separately cultured for about 12 hours in about 1 ml RPM1 media containing about 10% fetal calf serum in a 12-well plate. Normal, HIV or ESRD IgM is then added to each of the GHOST CCR5 and CXCR4 cells about 30 minutes prior to adding the R5 HIV-1 virus to GHOST CCR5 and the X4 HIV-1 virus to GHOST CXCR4. Both virus and antibody are present throughout the 48-hour culture period. No polybrene is used to enhance viral entry into the cells.
   After the 48-hour incubation period, cells are harvested and fixed in formalin. Infected cells emitting green fluorescence are enumerated with flow cytometry. Additionally, similar data is obtained when the virus or IgM antibody is washed about 4 hours after incubating with GHOST cells.
b) HIV Infection of Activated Human PBL
   Human PBL are pre-treated with Phytohemmaglutium (PHA-P) and IL-2 to increase receptor expression (e.g. CCR5, CD4) on T lymphocytes and monocytes as well as to activate such cells, both of which enhance HIV-1 entry and replication. Therefore, Ficol/Hypaque separated PBL (2×10⁶ cells per ml in culture media containing 10% fecal calf serum) are pretreated with PHA-P (5 mg/ml) and IL-2 (40 units/ml) and cultured for 24 to 48 hours in 5% CO₂. Cells are washed prior to adding IL-2, IgM and the HIV-1 virus. The cells are not washed any more but are kept growing for 12 to 14 days. On day 7, half the culture supernatant is removed (and saved) and the culture well is supplemented with 1×10⁶ freshly activated PBL (48 hour old) and also replenished with half the quantity of IgM and IL-2. On day 12 to 14 culture supernatants are harvested and p-24 core antigen in culture is quantitated using and ELISA technique
c) HIV-1 Infection of Human PBL/SCID Mice
   We employed (with modifications) the procedure developed by Mosier and as described in Torbett et al, Immunol Reviews 124: 139-164, 1991, which is incorporated herein by reference. Seven to eight week old female CB 17 SCID mice, purchased from Harlan Sprague Dawley, Indianapolis, Ind. and having <1 μg per ml of mouse IgM in their plasma were injected intraperitoneally with freshly isolated 25-35×10⁶ PBL in 1 ml RPMI containing 10% FCS and antibiotics (RPMI culture media). Two hours later mice were re-injected intraperitoneally with 10⁵ TCID₅₀ HIV-1 virus in 1 ml RPMI culture media. One ml of IgM at 1 mg/ml, obtained from the same PBL donor, was injected intraperitoneally, either immediately after the HIV-1 injection or 48 hours later. The same dose of IgM was injected every five days until day of sacrifice as kinetic studies revealed that human IgM in mouse plasma attained peak levels of 40-50 μg by day two and 8-10 μg per ml by day five after the intraperitoneal dose. Mice were sacrificed three weeks after the human PBL injection. Percent human CD3 and CD4 positive T lymphocytes in spleen cells were quantitated with FITC labeled mouse anti-human CD3 or CD4 (BD Pharmigen) using flow cytometric techniques. Secondly, murine spleen cells were co-cultured with two day old day IL2-activated autologous PBL to quantitate HIV-1 in spleen cells. In co-culture studies 2×10⁶ spleen leucocytes in 1 ml RPMI culture media were co-cultured with 2×10⁶ PHA+IL-2 activated (2 days old) human PBL in 1 ml RPMI culture media containing human IL2 (30 units/ml). Co-cultures were fed at weekly intervals with two-day-old 2×10⁶ IL2-activated autologous PBL. p24 antigen in co-culture supernatants was quantitated after two and three weeks of co-culture using an ELISA kit. With this protocol (i.e. single dose of virus and sacrifice at three weeks) one could not detect viremia after the first week. Studies on SCID mice were approved by our Institutions Animal Care and Use Committee.
      Results
      INTRODUCTION
      Data in the result section will be presented in the following order:
a) Studies to show that IgM binds to Lymphocytes, other leucocytes and malignant cells and studies to show that IgM does not cause complement mediated cell lysis at 37° C.
b) Studies to show that purified serum IgM inhibits HIV-1 infection (i) in-vitro and (ii) in-vivo.
c) Studies to show that IgM inhibits T cell proliferation and chemotaxis.
d) Studies to determine some of the mechanisms for IgM inhibition of T cell activation and proliferation including (i) irununoprecipitation studies to show that IgM binds to CD3 and CD4 and (ii) studies showing that IgM down-modulates CD4 and CD2 receptors, (iii) studies showing that IgM inhibits proximal intracellular events activated by the T_{c}R/CD3 receptor and (iv) studies showing that IgM inhibits secretion of certain chemokines and cytokines e.g. TNF-α, IL-13, MDC and TARC.
e) Studies to determine some of the mechanisms for IgM inhibition of chemotaxis including (i) immunoprecipitation studies to show that IgM binds to CCR5 and CXCR4, (ii) studies showing that IgM inhibits binding of MIP-1α and RANTES to CCR5 and inhibits binding of SDF-1α to CXCR4, (iii) studies showing that IgM down-modulates CCR5 but not CXCR4, and (iv) studies showing that IgM prevents chemokine induced internalization of CXCR4
f) Summary of above data delineating mechanisms for IgM mediated inhibition of HIV-1
g) Studies to show that IgM anti-lymphocyte autoantibodies inhibit the inflammatory response mediated by an allograft (i.e. rejection) in kidney transplant recipients.
h) Studies to show that IgM anti-lymphocyte autoantibodies cause cell death of lymphoma cells at 37° C.
   Presentation of Data
   a) IgM Binds to Lymphocytes, Other Leucocytes and Malignant Cells and Does not Cause Cell Lysis at 37° C.
(i) Binding of IgM to Lymphocytes, other Leukocytes and Malignant Cells
In these studies flow cytometric techniques were used to quantitate binding of IgM to the different cells. As seen in FIGS. 1A and 1B, Normal IgM, and HIV IgM contain IgM antibodies that bind to Sup T-1 (FIG. 1A) and GHOST CD4-CXCR4 cells (FIG. 1B). As seen in FIGS. 1D and IE, Normal and HIV IgM contains antibodies that bind to T-lymphocytes isolated from peripheral blood (FIG. 1D) and neutrophils isolated from peripheral blood (FIG. 1E). The negative control in each figure indicates that no IgM was incubated with the various cells.
(ii) Non-lytic Nature of IgM Anti-lymphocyte Antibodies at 37° C.
About 40 to 60% cell lysis of normal lymphocytes was observed in presence of complement when the assay was performed at 15° C. Higher levels of cell lysis was observed with IL-2-activated lymphocytes, which have increased expression of receptors. IgM, when used at amounts of about 1.0 microgram or more, caused cell lysis, while CK15 lysed cells at concentrations of about 0.05 microgram or more. When the assay was performed at 37° C., however, less than about 5% lysis was observed with normal or IL-2 activated lymphocytes. These observations are in agreement with several reports clearly demonstrating that IgM anti-lymphocyte autoantibodies are lytic at colder temperatures but not at 37° C. (See Lobo P I et al in Lancet Vol 2, p 879-83, 1980).
   b) Studies to Show that Purified Serum IgM Inhibits HIV-1 Infection of Human PBL
(i) In-vitro Studies to Show that Human IgM Inhibits HIV-1 Infection of PHA+IL-2 Activated Human PBL.

IgM, in these studies, were obtained from sera of normal individuals, HIV-1 infected individuals ESRD patients awaiting kidney transplantation. We did not have HIV-1 infected long term non-progressors that were on no HAART therapy. All the HIV-1 infected patients we studied were on HAART therapy. We used ESRD IgM to compare if IgM-ALA that develops as part of an inflammatory response in ESRD or after HIV-1 infection have similar inhibitory activities as predicted by our hypothesis. In these studies we also used IgM purified from serum of rats, mice, goats, and rabbits. In the initial studies different HIV-I strains (X4 and R5) were used to infect 48 hour mitogen activated PBL and using different concentrations of IgM, all in the physiological range. Maximal inhibitory activity was noted with all IgM preparations at 15 or more μg/ml although with certain viral strains near maximal inhibition was seen with IgM as low as 4 μg/ml. IgM from 4 to 5 individuals were pooled due to insufficient quantity but in certain experiments, (where indicated) IgM, from single individuals were used and there was no difference in the overall results. Data from 5 different experiments are presented in Table I and Table II using an R5 strain (8658) and the X4 strain IIIB. Interestingly, Normal, HIV, and ESRD IgM, as well as sol CD4 inhibited viral replication of the 8658 (R5) and the IIIB (X4) strains by more than 98%. All animal IgM, when compared to human IgM, had even more inhibitory effect on all the HIV-1 viral stains tested.

TABLE I
Effect of purified IgM from normal, HIV, and ESRD individuals on in-
vitro infectivity of HIV-1 virus

Pg/ml of p24 core antigen

| | IIIB(X4) | HIV-1 8658(R5) |
|---|---|---|
| Media | >32,813 | 34,602 |
| Normal IgM | 1,514 | 579 |
| HIV IgM | 439 | 672 |
| ESRD IgM | 870 | 230 |
| Waldenstrom IgM | >32,813 | 32,700 |
| Autologous Human serum | ND | 4,249 |
| RANTES (500 ng) | ND | 11,246 |
| Sol-CD4-183 (20 μg) | ND | 3,949 |
| pool Human IgG (50 μg) | >32,813 | ND |

Table I - Data are representative of 5 different experiments. Each p24 value is a mean of triplicate cultures with less than 15 percent variation from the mean. In these studies IgM used were from a pool of 3 to 4 different individuals and added 30 minutes before the virus.
ND = Not Done

TABLE II
Summary of all in-vitro studies evaluating percent inhibitory effect of
Normal, ESRD, and HIV-1 IgM on different HIV strains
Mean of Percent Inhibition

| | IIIB (X4) | 8658 (R5) |
|---|---|---|
| Normal IgM | (3) 97.6 ± 1.7 SD | (5) 98.6 ± 0.9 SD |
| ESRD IgM | (3) 99.2 ± 1.0 SD | (4) 99.1 ± 0.6 SD |
| HIV IgM | (3) 99.4 ± 0.5 SD | (4) 99.4 ± 0.9 SD |

(N) indicates number of different experiments, each done in triplicate. P-24 levels in viral cultures without IgM varied from 29,000 to 200,000 pg/ml

We next studied the kinetics of the inhibitory effect of IgM. As depicted in FIG. 2, purified normal IgM inhibited HIV-1, IIIB(X4) as well as 8658 (data not shown) even when added 96 hours after initiation of the viral cultures. These findings prompted us to determine if there was anti-viral activity in non-IgM-ALA antibodies. To exclude this possibility, IgM was initially absorbed with the Jurkat T cell line and the U937 monocytoid cell line to remove IgM with binding to CD3, CD4, CXCR4, and CCR5. As seen in Table III, the inhibitory activity of IgM on HIV-1 infectivity was removed after absorbing with the U937 and T cell line, thus indicating that the inhibitory activity on HIV-1 resides in IgM that binds to leucocytes (i.e. IgM-ALA). The data thus far suggested that IgM-ALA could inhibit HIV-1 by inhibiting viral entry. We resorted to the GHOST CCR5 and GHOST CXCR4 tranfectant cell lines to verify that IgM inhibits viral entry. These cell lines are stably co-transfected with the HIV-2 LTR driving hGFP construct, which emits a green fluroscent upon integration of HIV-1 viral genome into the cell DNA. Hence one can measure entry efficiency of the virus especially if cells are harvested in 48 hours, which allows for a single cycle of viral replication. Data from FIG. 3 clearly demonstrates that in the presence of IgM, viral entry is reduced by more than 95%.

TABLE III
Experiment to determine if the HIV-1 inhibitory activity in Normal pool
IgM resides in IgM that binds to T cells (i.e. IgM-ALA) - effect
of absorbing IgM with Jurkat
T cell line and U937 cells

p24 antigen (pg/ml)

| | IIIB(X4) | 8658(R5) |
|---|---|---|
| Media | 14,849 | 23,525 |
| Normal pool IgM | 2,134 | 581 |
| (16 μg/ml) | | |
| Normal pool IgM | 13,107 | 28,061 |
| (16 μg/ml) absorbed | | |
| with Jurkat and | | |
| U937 cells | | |

Table III - Experimental details as in Table I. IgM was added on Day 0. Details of IgM absorption are in section of “Methods of Procedure”. Representative data from two separate experiments are presented. Data are mean of triplicates with less than 10 percent variation from the mean

(ii) Studies to Show that Normal IgM Inhibits In-vivo HIV-1 Infection in a Human PBL-SCID Mice Model

We used this well described in-vivo model to confirm observations with the in-vitro PHA+IL-2 activated PBL assay. The PBL in this model are not pre-activated with mitogen prior to viral infection and hence the inhibitory effect of IgM-ALA on T cell activation can also play a role in controlling viral replication. Details of the experimental method and quantitation of IgM levels in the serum are described in section on “methods of procedure”. Studies were not done with HIV and ESRD IgM as it was difficult to obtain blood in quantities needed for these experiments. Data with pooled normal IgM and the two different HIV strains are depicted in Table IV. These data bring out two observations. Firstly, 30 percent of infected mice can spontaneously become non-infected because of CD4 cell depletion, and this observation was also noted by Mosier. Hence at 3 weeks 60-70% of mice remained infected. However, normal IgM reduced the number of infected mice to 27% with the 8658 (R5) strain and 14% with the IIIB(X4) strain. This decrease in infected mice in the presence of normal human IgM was statistically significant (p<0.05, Fishers Exact Test) when one combined data of both 8658 and IIIB viral strains. The decrease in HIV-1 infection of human-PBL-SCID mice in the presence of human IgM was not due to IgM or HIV-1 depletion of human PBL as by three color flow cytometry we could not detect significant changes in the splenic human T cell population (CD45+, CD3+, CD4+) between SCID mice treated with IgM+HIV+PBL and control SCID mice treated with PBL (data not shown).

TABLE IV
Experiments to determine if normal pool IgM inhibits X4 and R5 HIV-1
viral strains in an in-vivo human PBL-SCID mice model.

| | # of mice infected at 3 weeks | |
|---|---|---|
| | 8658(R5) HIV-1 virus | IIIB(X4) HIV-1 virus |
| PBL | 0/4 | 0/4 |
| PBL + HIV | 10/15 (66%) | 3/4 (75%) |
| PBL + HIV + IgM | 3/11 (27%) | 1/7 (14%) |

In summary, these data clearly showed that IgM obtained from normal, ESRD, and HIV-1 infected patients inhibits HIV-1 from infecting activated human PBL in-vitro and in-vivo and this inhibitory effect is removed after absorbing IgM with the U937 monocytoid line and the Jurkat T cell line indicating that inhibition of HIV-1 infectivity is mediated by IgM that binds to the cell membrane of leucocytes. Additionally, experiments with the GHOST cells indicate that the inhibitory effect of IgM is mediated by decreasing efficiency of viral entry. Our findings cannot be explained, on IgM with reactivity to Tat and gp120, which may be present in the purified IgM preparations as previous investigators have shown that IgM with anti-Tat and anti-gp120 do not have HIV-1 neutralizing activity and do not inhibit viral entry into cells. Similarly, our findings cannot be explained on IgM neutralizing the HIV-1 virus as there is prior art to show that fresh human serum does not lyse or inactivate the HIV-1 virus (see Rodman T C et al, J of Exp Med, Vol 175 p1247-1353, 1992; Berberian et al Science Vol 261 p 1588-1591, 1993; Llorente M. Scand J of Immunol, Vol 50 p270-279, 1999; Hoshino H, Nature Vol 310 p324-325, 1984; and Bonapur B, Virology Vol 152 p 268-271, 1986 for prior art in this regard. We could not detect RANTES or SDF-1α in these IgM preparations using ELISA and Western blot techniques.

The increase in IgM-ALA to diverse inflammatory processes and the inhibition by IgM-ALA of HIV-1 infectivity prompted us to evaluate whether IgM-ALA mediates this inhibitory effect by binding to receptors needed by the HIV-1 virus for cell entry as well as receptors involved in inflammation. Binding of IgM to T cell receptors and to chemokine receptors appeared to be an attractive possibility. We initially examined these possibilities by determining if IgM purifiedfrom serum (i) inhibited alloantigen (MLR) and anti-CD3 induced T cell proliferation and (ii) inhibited chemotaxis in response to chemokines. In these studies, we compared normal IgM with HIV-1 and ESRD IgM. Waldenstrom IgM was used as a negative control in these studies.

c) Studies to Show that IgM Inhibits T cell Proliferation and Chemotaxis

(i) IgM Inhibits MLR-induced Proliferation
   An MLR assay (see methods) was used as an initial step to evaluate the effect of IgM on T cell proliferation in response to alloantigens. As can be seen from FIG. 4, pooled ESRD IgM, but not pooled normal and HIV IgM, significantly inhibited T cell proliferation using physiological doses of IgM i.e. 15 μg/ml. ESRD IgM failed to inhibit T cell proliferation when added after 24 hours of culture. Pooled IgG or albumin had no inhibitory effect in the MLR assay. Normal IgM inhibited MLR when used at 40 to 60 [symbol] g/ml (data not shown).
   To determine if the observed effect of ESRD IgM was due to IgM that bound to T cells, we absorbed ESRD IgM with the U937 and Jurkat T cell line (see methods) to remove any IgM anti-leucocyte reactivity. IgM absorbed with these cell lines failed to inhibit T cell proliferation in the MLR assay clearly indicating that the observed inhibition of T cell proliferation with ESRD IgM was due to IgM that bound to leucoyctes.
(ii) IgM Inhibits Anti-CD3 Induced T cell Proliferation
   We wanted to determine if IgM affects anti-CD3 induced proliferation of PBL. In these studies normal PBL (3×10⁵ in 0.3 ml) were exposed to 0.01 μg OKT3 (a murine IgG2a anti-CD3 monoclonal) and then cultured for 4 days in 96 well flat bottom plates prior to determining extent of cell proliferation using H ³-labeled thymidine. Pooled normal, HIV IgM, or ESRD IgM (15 μg) was added to these cell cultures at initiation of the culture. Data from one of 3 experiments is depicted in FIG. 5. HIV and ESRD IgM significantly suppressed anti-CD3 mediated proliferation of T cells. Again ESRD IgM failed to inhibit T cell proliferation when added after 24 hours of culture. These data are similar to those observed with the MLR induced T cell proliferation (See FIG. 4).
(iii) IgM Inhibits Chemotaxis
   We wanted to determine if IgM inhibits chemotaxis of activated PBL and T cell lines in response to chemokines. All IgM preparations inhibited chemotaxis. However ESRD IgM had a significantly more pronounced inhibitory effect on chemotaxis as depicted in FIG. 6A for HuT78 and 6B for the Jurkat T cell line.
   These differences in inhibitory effects on chemotaxis with the T cell lines were not due to increased apoptosis or cell death as evaluated by flow cytometry using propridium and anti-annexin and would suggest that ESRD IgM in addition inhibits chemotaxis through effects on other cell receptors (e.g. adhesion molecules or integrins) and/or intracellular activation pathways that are involved in both chemokinesis and chemotaxis activity. Data in FIG. 6A and 6B shows that both normal and ESRD IgM has an inhibitory effect on chemokinesis of cells in the absence of SDF-1α. However, ESRD IgM has a more pronounced effect on chemotaxis when compared to normal IgM, suggesting that ESRD IgM may in addition inhibit intracellular activation pathways involved in chemotaxis.
      d) Studies to Determine Mechanism for IGM-ALA Inhibition of T cell Proliferation
   Inhibition, especially by ESRD IgM, of T lymphocyte proliferation in response to alloantigens or anti-CD3 prompted us to determine if the inhibitory effect mediated by IgM was secondary to binding of IgM to TcR/CD3 and/or the co-stimulatory molecules. IN support of such a concept are studies showing that binding of antibodies to the CD4 receptor, inactivates T cell proliferation in response to alloantigens or anti-CD3. Additionally there are studies to show that binding of antibody to CD3 (e.g IgG anti-CD3) inhibits T cell proliferation in response to alloantigens (MLA). We also wanted to determine if binding of IgM to the receptor resulted in down-regulation of the receptor. In these studies we used IgM purified from individual normal sera and compared to IgM obtained from individual HIV and ESRD IgM. These purified IgM preparations were used to immunoprecipitate different receptors from whole cell lysates of cell lines constitutively expressing high levels of these receptors.
(i) Immunoprecipitation Studies Showing that IgM Binds to CD3 and CD4
   Here receptors in whole cell lysates were immunoprecipitated with purified individual normal, HIV or ESRD IgM, and then subjected to SDS-PAGE gel electrophoresis under reducing conditions at 37° C. for 30 minutes with 2ME (see methods for details). Receptors immunoprecipitated by IgM were transferred on to nitrocellulose membranes prior to using murine monoclonal or rabbit IgG polyclonal antibodies as primary antibodies to identify these receptors. We used several controls to exclude the possibility of non-specific receptor binding to the bead (i.e. in absence of IgM).
   Representative data from 3 separate experiments involving identical quantities of normal, HIV IgM, and ESRD IgM as well as identical quantities of whole cell lysates are depicted in FIG. 7. The data clearly demonstrates that both normal, HIV, and ESRD IgM immunoprecipitated CD3 and the CD4 receptor. As a group, HIV-IgM appeared to immunoprecipitate more CD4, when compared to Normal or ESRD IgM. Waldenstrom IgM (labeled W) did not immunoprecipitate CD4.
   We next wanted to determine if inhibition of proliferation by IgM was merely due to IgM binding to CD3 and CD4 (thus causing a perturbation in the formation of the immunological synapse) or did IgM in addition down-modulate the receptors especially in light of previous studies showing that cross-linking of CD3 can down-regulate CD4.
(ii) Studies to Show that IgM Down-regulates CD4, CD2, CD86 but not CD8, HLA, and other Co-stimulatory Molecules
   In these studies we used the MLR assay to activate T cells. Different doses of normal IgM were added either at the initiation of MLR, on day 3 of culture or 2 hours prior to harvesting the cells on day 4 of culture. Day 4 MLR activated cells were analyzed using two color flow cytometry for T cell co-stimulatory molecules. We used either PE or FITC-labeled murine monoclonals specific for the different receptors. Representative data from 4 different experiments involving different combinations of individuals are depicted in FIG. 8. We noted that normal, HIV, and ESRD IgM, when added to MLR cultures, markedly inhibited the density of certain co-stimulatory molecules on the cell membrane e.g. CD4 and CD2 but had no effect on CD3, CD 28 and CD8 (FIG. 8). HIV, ESRD, and Normal IgM did not, however, down-regulate CD154, CD28, CD3, PDL-1, IL2-R, HLA-A, B, HLA-DR membrane receptors, as well as surface and intracytoplasmic CD152 receptors (data not shown). Other studies were performed to determine if IgM inhibits expression of co-stimulating molecules i.e. CD80 (B7.1) and CD86 (B7.2) present on antigen presenting cells. In these studies, we evaluated CD80 and CD86 expression on CD14 positive monocytes and macrophages present in the MLR assays except receptor density was evaluated at 24 hours of initiating the MLR culture. IgM markedly inhibited expression of CD86 (but minimally inhibited expression of CD80) on CD14 positive monocytes and macrophages as exemplified in FIG. 9 which depicts IgM inhibiting ESRD IgM on expression of CD86. This inhibitory effect was not accompanied by increased apoptosis or cell death as measured by flow cytometry quantification of annexin expression and propidium iodide uptake by cells. The degree of inhibition for CD4 and CD2 was similar whether IgM was added on Day 0 of MLR or 2 hours before termination of the MLR culture. Secondly, there was no significant difference in level of inhibition between normal or HIV or ESRD IgM when used at doses varying from 10 to 30 μg/ml. No inhibition was observed at doses less than 5 μg/ml.
   Further experiments were performed to investigate the mechanism for the inhibitory effect on CD4 and CD2. Firstly we wanted to determine whether the inhibitory effect in the presence of normal or HIV IgM was an “active” process or due to a “blocking” effect i.e. by IgM inhibiting the binding of the murine anti-receptor monoclonal antibody that is used to detect the receptor. IgM was added 2 hours prior to termination of MLR on Day 4 except an aliquot of cells was also incubated at 4° C. with IgM during the 2 hour period. In 3 separate experiments, there was no decrease in MCF of co-stimulatory receptors when IgM was incubated with cells at 4° C. indicating therefore that the decrease in density of surface co-stimulatory receptors was due to an “active” process. Either there was internalization of receptors or active down-modulation of receptors at 37° C. in the presence of IgM. This question was analyzed using flow cytometry. In these studies, we focused mainly on CD4 expression as these receptors were highly expressed. Cells were initially exposed to PE-anti CD4 to stain for surface receptor and after washing the cells were permeabilized using the BD Pharmigen Kit and then re-exposed to PE-anti CD4 to stain for intracytoplasmic receptors. Data are presented in FIG. 10. Data indicates that IgM at 37° C. down-regulated both surface and intra-cytoplasmic CD4 receptors.
   We next wanted to determine if down-modulation of both membrane and intracytoplasmic CD4 was secondary to cross-linking of CD3 by the pentameric IgM or possibly a direct effect secondary to binding of IgM to CD4. Two approaches were used. Firstly we used a human monocytoid cell line (U937) which expresses CD4 but has no CD3 receptor. Incubating U937 cells for 2 hours at 37° C. in presence of normal or HIV IgM led to a 50 to 55% reduction in expression of CD4 indicating that down-modulation of CD4 by IgM was independent of CD3. Secondly, MLR activated lymphocytes were incubated at 37° C. for 2 hours with either pentameric or monomeric IgM. Again use of monomeric HIV IgM led to down-modulation of CD4 indicating that cross-linking of the CD4 receptor was not essential for down-modulation.
(iii) IgM-ALA Inhibits Proximal Signaling Events Involved in in T cell Activation
   Prior studies have shown that T cell activation mediated by TcR pertubation results in recruitment, phosphorylation and activation of Zap 70 (see Pullar C E, Scand J of Immunol, Vol 57, p333-341, 2003 for prior art in this regard). We therefore, wanted to determine if IgM inhibits phosphorylation of Zap 70 induced by anti-CD3.
   In these studies freshly obtained human peripheral blood lymphocytes (1×10⁶ cells/ml) were pretreated with immobilized anti-CD3 for 12 hours at 37° C. in 5% CO₂ and then examined for intra cytoplasmic phosphorylation of Zap 70 using flow cytometry. Intracytoplasmic phospho Zap 70 was quantitated by fixing and permeabilising the cells prior to interacting the cells with a polyclonal rabbit antibody to phospho Zap 70 (Cell Signalling, MA.). Purified IgM (30 μg/ml) from normal, HIV and ESRD patients was added to the cells half an hour prior to adding the cells to immobilized anti-CD3.
   As can be seen in FIG. 11, there was increased phosphorylation of Zap 70 in human T cells activated with anti-CD3. However, pretreatment of T cells with normal or HIV IgM inhibited Zap 70 phosphorylation.
(iv) IgM Inhibits Secretion of TNF-α, IL-13, MDC and TARC
   Further studies were performed to determine if the anti-proliferative effects of IgM-ALA were associated with a decrease in cytokine production. Supernatants from MLR cultures (Day 5 to 6) were assayed for different cytokines in a semi-quantitative manner using the Array III kit, which can detect cytokines in culture media at levels of 5 to 10 pg/ml (see methods for details). The Array III kit detected a significant increase in the secretion of IL-6, IL-8, IL-13, TNF-α, GMCSF, MCP-1, MIG, MDC, TARC, and GRO in the MLR supernatants. However, presence of IgM at the initiation of the MLR culture had no inhibitory effect on production of IL-6, IL-8, GMCSF, MCP-1, MIG, and GRO (see FIG. 3D). Conversely all IgM preparations, including normal IgM, significantly inhibited secretion of TNF-α, IL-13, MDC, and TARC (see FIG. 12). Inhibition of TNF-α is particularly important as prior art has shown that inhibitors of TNF-α (e.g. antibodies to TNF-α) can suppress inflammation in patients with rheumatoid arthritis and Crohn's disease (see Feldman M, Annual Rev Immunol 2001, vol 19, p 163-196; and Sandborn W J Inflamm Bowel Dis., 1999 vol. 5 p 119-133 and the material in these references is incorporated herein by reference). The changes in cytokine levels were similar whether supernatants were assayed on Day 1,2, or 3 of the MLR culture. Cytokine levels were maximal on Day 5 of MLR as exemplified for TNF-α in FIG. 12. No IL-2, INF-γ, TGF-β, and IL-10 could be detected in the MLR supernatants using the Array III assay technique.
   These data provide more evidence indicating that IgM-ALA can inhibit T cell function in addition to proliferation.
   In summary, normal, HIV, and ESRD IgM immunoprecipitate CD3 and CD4 receptors. IgM-ALA also mediates CD4 and CD2 receptor down-modulation, independent of CD3 and in addition IgM inhibits phosphorylation and activation of Zap 70 which are important for T cell activiation. IgM in addition, inhibits secretion of certain cytokines—in particular TNF-α, IL-13, MDC and TARC. All these mechanisms most likely contribute to IgM-mediated (i) inhibition of T cell activation and proliferation induced by alloantigenic stimuli (MLR) or anti-CD3 antibodies, and (ii) inhibition of HIV-1 infectivity of cells.
      e) Studies to Determine Mechanisms for IgM-ALA Mediated Inhibition of Chemotaxis
   In these studies we wanted to determine if inhibition of chemotaxis was secondary to IgM-ALA down-modulation of these receptors (from inhibition of T cell activation) or due to a direct “blocking” effect of IgM-ALA on the binding of chemokine to the receptor.
(i) Immunoprecipitation Studies to Show that IgM Binds to CCR5 and CXCR4
   Initially, we wanted to determine whether IgM bound to the chemokine receptor. We approached this question by determining whether IgM could immunoprecipitate CCR5 and/or CXCR4 from whole cell lysates of the Daudi B cell line, which constitutively expresses high levels of CCR5 and CXCR4. Representative data from three separate experiments, using identical quantities of IgM and whole cell lysates from three different normal individuals, pooled normal IgM (6 individuals), pooled ESRD IgM from 5 individuals, and five individual HIV IgM is depicted in FIG. 13. As depicted in FIG. 13, all three normal IgM individuals immunoprecipitated low levels of CCR5 while only one of five HIV individuals immunoprecipitated CCR5 suggesting that HIV-IgM, unlike normal IgM, has decreased IgM with binding reactivity to CCR5. ESRD IgM, on the other hand, immunoprecipitated severalfold more IgM anti-CCR5 when compared to Normal IgM. Immunoprecipitation studies with CXCR4 were totally unexpected. Here four of the five HIV IgM and all of the ESRD IgM had IgM with a high level of binding reactivity to CXCR4. In summary, different individuals, whether normal or with disease, produce different levels of IgM with reactivity to CCR5 or CXCR4. Interestingly, disease processes can also alter IgM anti-CCR5 or anti-CXCR4 profile. HIV-1 infected individuals, in general, lack IgM anti-CCR5, while ESRD individuals produce high levels of IgM with reactivity to both CCR5 and CXCR4. Waldenstrom IgM (labeled W) failed to immunoprecipitate CCR5 or CXCR4.
   The lane containing only lysate (Ly) in FIG. 13 clearly demonstrates that Daudi lysates contain the non-glycosylated 36-39 kDa isoform of CXCR4, which is expressed at high levels on the cell membrane and detected by the 4G10 and 12G5 murine monoclonals. No glycosylated 47 kDa isoform of CXCR4 was present in the Daudi lysate. Note, however, that Daudi lysate contained the glycosylated isoform of CCR5 (42-43 kDa) which was immunoprecipitated by IgM.
(ii) IgM Inhibits Binding of MIP-1α and SDF-1α to their Receptors
   Since IgM immunoprecipitated CXCR4 and CCR5 from cell membranes, it became important to determine if IgM inhibited binding of chemokine to these receptors. Data in FIG. 14 clearly demonstrates that both Normal and ESRD IgM inhibited to a similar degree binding of biotin labeled MIP-I α to CCR5 and SDF-I α to CXCR4 present on two cell lines and on PBL activated for 3 days with PHA and IL-2. IgM inhibited chemokine binding in a dose dependent manner as exemplified for binding of MIP-1α to U937 cells, SDF-1α to Hut-78 cells and SDF-1α to activated PBL. Incubating cells with IgM and/or chemokine at 37° C. or 40 C. did not change the magnitude of the inhibitory effect of IgM on chemokine binding thus indicating that the IgM mediated inhibitory effect was not due to internalization of the receptor at 370° C. Waldenstrom IgM and pooled human IgG had no inhibitory effect on chemokine binding.
(iii) Studies to Show that IgM Prevents Internalization of CXCR4.
   Ligands that bind to chemokine receptors induce receptor internalization. Such a process occurs after binding of chemokines or HIV-1 to the receptor. If therefore became important to determine if IgM, after binding to the chemokine receptor, induces receptor internalization. This question was investigated by determining whether IgM induced CXCR4 internalization after binding to the receptor or in the presence of SDF-1α. In these studies we used a murine IgG anti-CXCR4 monoclonal (e.g. 12G5) that does not compete with IgM for the same binding sites on the CXCR4 receptor. To study this question, Jurkat T-cells expressing CXCR4 were pretreated with ESRD IgM (pre absorbed with mouse IgG) at 37° C. for 30 minutes, not washed, and then cells were interacted with SDF-1α (100 μg) at 37° C. for another 30 minutes . Cells were then washed and interacted with FITC labeled 12G5 to detect CXCR4 expression. Data in FIG. 15 (panel B) clearly indicates that SDF-1α markedly reduces CXCR4 expression at 37° C. (secondary to internalization) in absence of IgM. However, pretreatment of cells with IgM (15 μg/10⁶ cells) at 37° C. does not lead to CXCR4 internalization (panel A) and in addition IgM inhibits CXCR4 internalization that occurs in presence of SDF-1α (panel B). Similar data were obtained with a SupT-1 T cell line and the RAJI B cell line.
   In summary, IgM-ALA (i) down-modulates CCR5 receptor expression, but not CXCR4 receptor expression, (ii) strongly inhibits RANTES and MIP-1α binding to CCR5 and also inhibits SDF-1α binding to CXCR4, and (iii) binds to both CCR5 and CXCR4 receptors except there are major differences in the level of IgM anti-CCR5 and anti-CXCR4 among different individuals and between disease states i.e. HIV-IgM from most patients have decreased IgM anti-CCR5 but not anti-CXCR4 while ESRD IgM has high levels of IgM reactive to both CCR5 and CXCR4. These observations provide a mechanism for IgM mediated inhibition of HIV-1 infectivity and for inhibition of leucoyte chemotaxis.
      f) SUMMARY: Delineating Some Mechanisms for IgM Mediated Inhibition of HIV-1 Infectivity
These data highlight certain observations:
   (i) IgM-ALA bind to CD3, CD4, CCR5, and CXCR4. However, there are major differences in the repertoire of IgM-ALA among individuals and between normal and disease states. For example, IgM from most normal individuals has low level of antibodies that bind to CCR5 and CXCR4 while many (but not all) HIV-1 infected individuals, have high levels of IgM with reactivity to CXCR4 and low levels of IgM with reactivity to CCR5. Conversely, ESRD IgM has high levels of antibodies to both CXCR4 and CCR5.
   (ii) IgM-ALA (a) inhibits T cell proliferation in response to alloantigens and anti-CD3 antibodies, with ESRD IgM having the most inhibitory activity, (b) significantly down-modulates CD4, CD2, CD86, and CCR5 receptors (but not CD8, CD3 and CXCR4) and again ESRD IgM has the most down-modulating effect on these receptors.
   (iii) IgM-ALA inhibits T cell activation as evidenced by decreased phosphorylation of Zap-70 and in addition IgM-ALA inhibits secretion of certain chemokines and cytokines, in particular TNF-α, IL-13, MDC and TARC.
   (iv) IgM-ALA in physiological doses, inhibits HIV-1 infectivity of PBL both in-vitro and in-vivo. This inhibitory effect of IgM on HIV-1 appears to be mediated by an inhibitory effect on viral entry (see GHOST cell experiments—FIG. 3) as well as on T cell activation. ESRD IgM which has high levels of IgM binding to CD4, CCR5, and CXCR4 has the most inhibitory effect.
      g) IgM Anti-lymphocyte Auto Antibodies Inhibit Rejections in Kidney Transplant Recipients.

Since normal IgM inhibited the binding of chemokines (SDF-1α and RANTES) to their respective receptors and since ESRD IgM inhibited lymphocyte activation in a mixed lymphocyte culture (MLC), it became necessary to test whether in-vivo, there would be a strong correlation between the presence of high levels of these antibodies in the recipient and protection against kidney transplant rejections.

Accordingly, the level of IgM anti-lymphocyte antibody activity in the recipient was quantitated using flow cytometry to detect binding of IgM to donor T lymphocytes (see FIG. 16). Presence of high IgM binding to donor CD3 positive T lymphocytes would also indicate that a similar level of IgM binding would occur with autologous leucocytes and donor endothelial cells.

TABLE V
Correlating quantity of recipient IgM binding to CD3 positive donor T
lymphocytes with human kidney transplant outcome

| | No IgM | LOW IgM | HIGH IgM |
|---|---|---|---|
| | (MCF < 20) | (MCF 21-200) | (MCF > 200) |
| # of Patients | 65 | 22 | 21 |
| % Acute Rejections | 32 | 32 | *9.5 |
| Requiring Treatment | | | |
| % Graft Loss | 20 | 9.1 | *0 |
| (1 year) | | | |

MCF = Mean Channel Fluorescence
*These data when compared to No and Low level Igm are statistically significant. (p < 0.02)

Data in FIG. 16 and Table V clearly shows that the presence of low or high IgM anti-lymphocyte activity as quantitated by mean channel fluorescence (MCF) was clearly associated with significantly less rejections and less graft loss at one year. All patients in this study were given the same immunosuppressive agents.

According to the present invention, the inventor believes that IgM anti leucocyte antibodies mediate protection against rejections by binding to autologous leucocytes (thus inhibiting chemotaxis of leucocytes and lymphocyte activation) and receptors on donor endothelial cells. The inventor has prior art clearly demonstrating that certain kidney recipients have IgM in their serum that binds to both donor lymphocytes and kidney endothelial cells. These data are described in Lobo et al, Lancet 2: 879-83, 1980 and the material in this reference is incorporated herein by reference.

h) IgM Anti-lymphocyte Antibodies Cause Apoptosis of Lymphoma cells at 37° C.

Malignant T lymphocytes, unlike normal IL-2 activated lymphocytes, undergo apoptosis in presence of IgM at 37° C. In these studies, we added 5 to 10 microgm of normal pooled IgM to 0.5×10⁶ Jurkat or Sup T-1 lymphocytes in 0.5 ml of RPM1 with 2% albumin. After thirty to 45 minutes incubation at 37° C. in 5% CO₂ cells were examined for apoptosis with anti-annexin antibodies and flowcytometry. No exogenous complement was added. Twenty to 35% of Jurkat or Sup T-1 cells were found to be dead under these conditions. There was less than 5% cell death of normal human lymphocytes or IL-2 activated lymphocytes when cultured under these conditions.

### BRIEF DESCRIPTION OF THE DRAWINGS

FIG. 1A is a flow cytometry histogram depicting binding of Normal IgM, HIV IgM and AIDS IgM to Sup T-1 cells.

FIG. 1B is a graph depicting binding of Normal IgM, HIV IgM and AIDS IgM to GHOST CXCR4 cells.

FIG. 1C is a flow cytometry dot plot showing lymphocytes and neutrophils separated by size and derived from human blood.

FIG. 1D is a flow cytometry histogram depicting binding of Normal IgM to human T lymphocyte derived from peripheral blood cells.

FIG. 1E is a flow cytometry histogram depicting binding of AIDS IgM to human neutrophils derived from peripheral blood cells.

FIG. 2 is a bar histogram showing that Normal IgM will inhibit HIV-1 IIIB infection of human PBL even when IgM is added to cells 4 days after HIV-1 infection of cells

FIG. 3 is a flow cytometry dot plot depicting that normal IgM will inhibit (i) HIV-1 (R5) 8658 viral strain from infecting GHOST-CCR5 (upper panels) and (ii) HIV-1 (X4) IIIB viral strains from infecting GHOST-CXCR4 (lower panels)

FIG. 4 is a bar histogram depicting that IgM, especially HIV and ESRD, inhibits proliferation of peripheral blood lymphocytes (PBL) activated in an MLR.

FIG. 5 is a bar histogram depicting that IgM, especially HIV and ESRD, inhibits proliferation of T lymphocytes activated by anti-CD3 antibody

FIG. 6 are bar histograms depicting that IgM, especially ESRD IgM, inhibits SDF-1α induced chemotaxis of HuT 78 (upper panel) and Jurkat (lower panel) malignant T cell lines as well as chemokinesis of cells (see bars shaded grey) in absence of SDF-1α.

FIG. 7 is a western blot to show differences in immunoprecipitation of CD3e and CD4 by different individual normal (labeled N,1,2, etc.), individual HIV (labeled H) and individual ESRD (labeled E) IgM from whole cell lysates of Jurkat cells. Ly and (Ly +B) are control lanes with only lysate (Ly) or lysate mixed with bead (Ly+B) but without IgM.

FIG. 8 are flow cytometry histograms depicting that ESRD IgM inhibits membrane expression of CD4, and CD2 but not CD8 and CD28. The shaded histogram represents receptor expression (quantitated by mean channel fluorescence—MCF) in absence of IgM

FIG. 9 is a flow cytometry histogram depicting that ESRD IgM, but not normal IgM, inhibits the co-stimulatory molecule CD86, on macrophages activated in an MLR for 24 hrs. The shaded histogram represents receptor expression in absence of IgM.

FIG. 10 depicts flowcytometry dot plots to indicate that normal IgM, but not control Waldenstrom IgM, inhibits CD4 expression on cell surface of T cells activated in a 3 day MLR (left panels) as well as intracytoplasmic CD4.

FIG. 11 Panel A depicts flowcytometry dot plots to show that ESRD and Normal IgM inhibits background phos-Zap-70 (shaded grey) in PBL as well as the increase in phos-Zap-70 following 16 hours of activation with anti-CD3 (OKT3). Panel B are bar histograms to show that all the different IgM (4 different HIV, one pooled ESRD, one Normal IgM) but not control Wadenstrom IgM, inhibited the increase in phos-Zap-70 after 16 hours of anti-CD3 activation. Data also depicts total Zap-70 (shaded bars) which did not increase with anti-CD3.

FIG. 12 depicts a radiograph of different human cytokines detected (using the Ray Bioassay kit) in supernatants of 6 day MLR performed in presence or absence of different IgM preparations. Note that all the different IgM, but not control Waldenstrom IgM, significantly inhibited production of TNF-α and IL-13.

FIG. 13 are western blots depicting differences in immunoprecipitation of CXCR4 and CCR5 by individual normal IgM (labeled N 1 or 2), pooled normal IgM (labeled N-P), pooled ESRD IgM (labeled E-P), individual HIV-1 IgM (labeled H 1 or 2, etc) and Waldenstrom IgM (labeled W). Lanes labeled Ly or Ly+B are similar controls as in FIG. 7.

FIG. 14 are graphs decpicting that normal and ESRD IgM, but not control Waldenstrom IgM, inhibits binding of SDF-1α to HuT-78 cells (upper panel) and MIP-1α binding to HuT 78 cells (lower panels).

FIG. 15 are flow cytometry histogram of Jurkat Cells depicting that ESRD IgM does not internalize CXCR4 (Panel A) but ESRD IgM will prevent internalization of CXCR4 receptor induced by SDF-1α (Panel B).

FIG. 16 depicts flow cytometry dot plots to show that different kidney transplant recipients have in their serum different quantities of IgM binding to their donor CD3 positive T lymphocytes. The lower dot plots depict binding of IgM to donor T lymphocytes after adding sera obtained from different recipients. Some recipient sera have no IgM anti-T lymphocyte antibody (left panel) while other sera have very high IgM anti-T lymphocyte antibody (right panel) as quantitated by mean channel fluorescence (MCF).

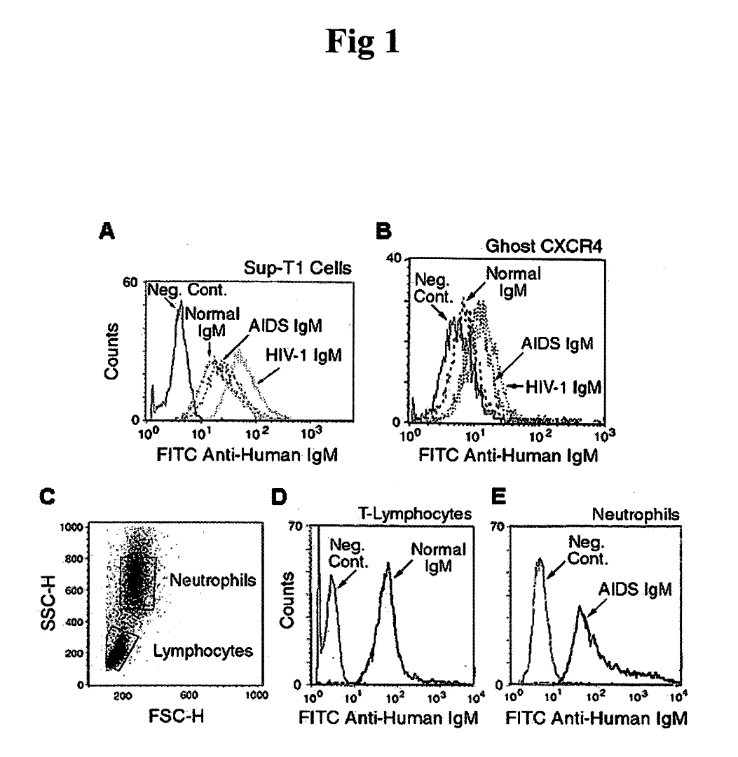

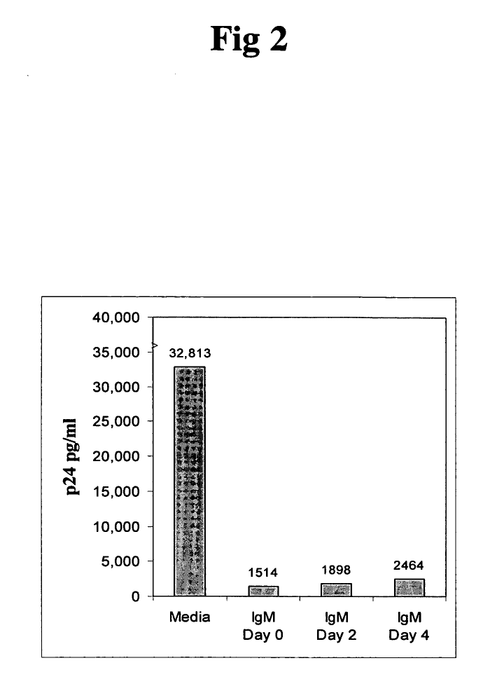

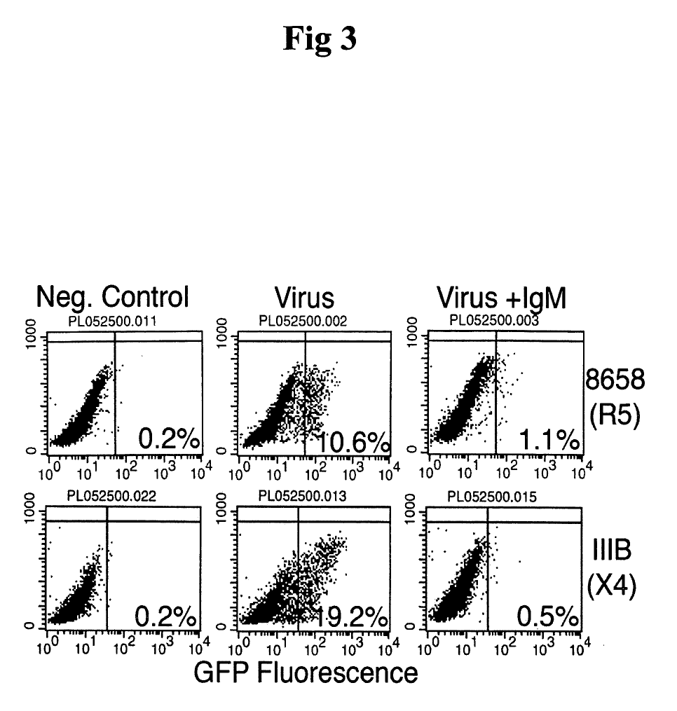

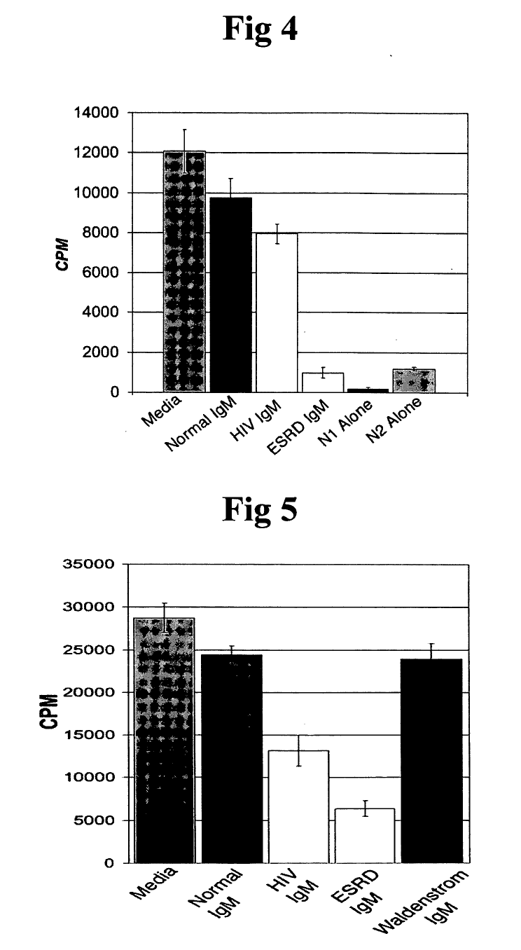

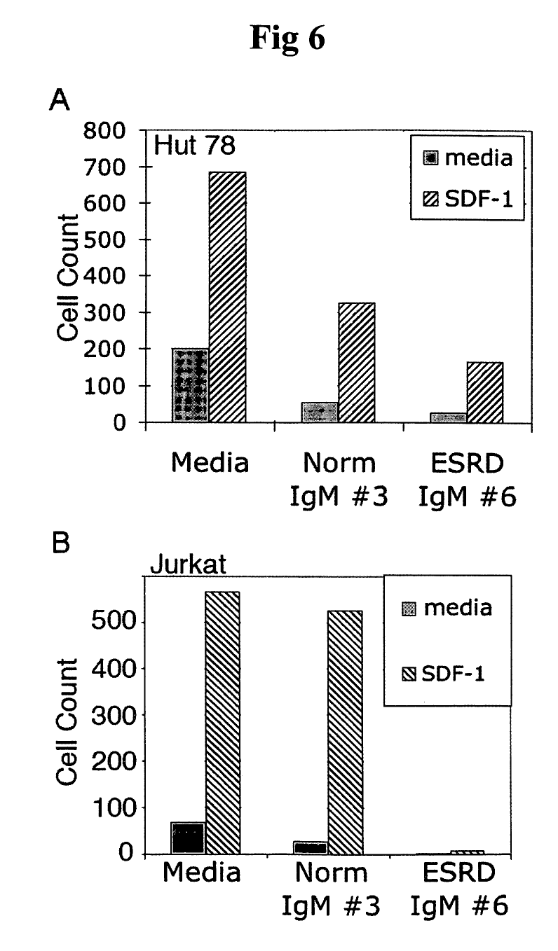

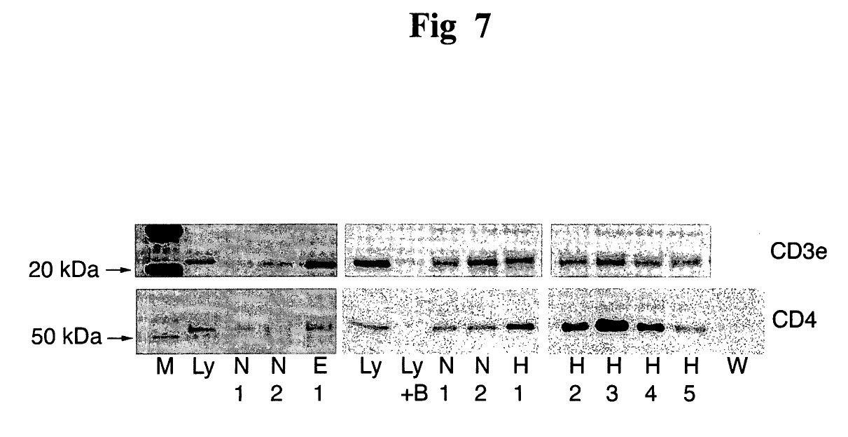

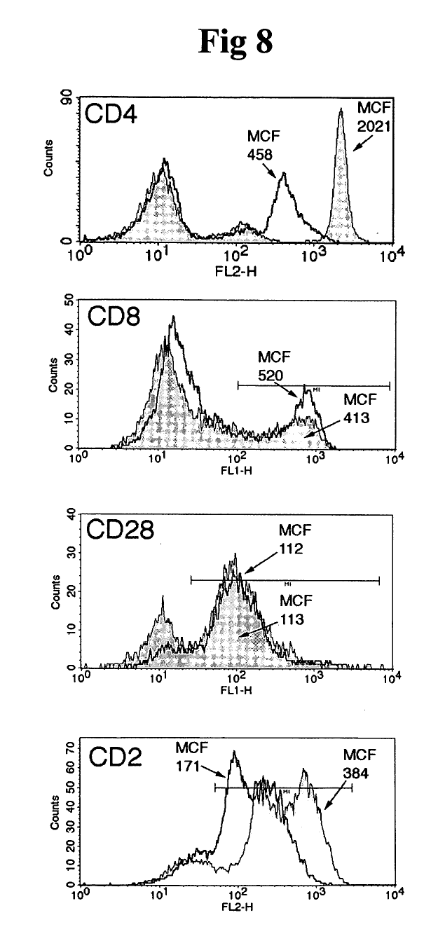

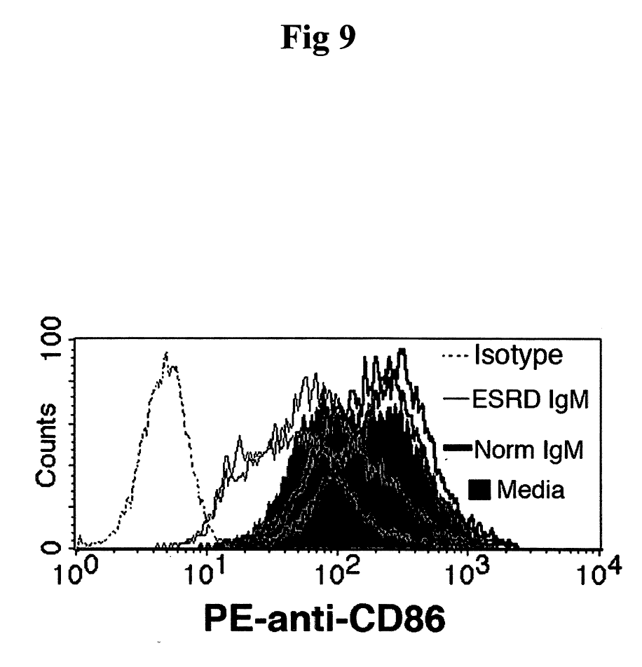

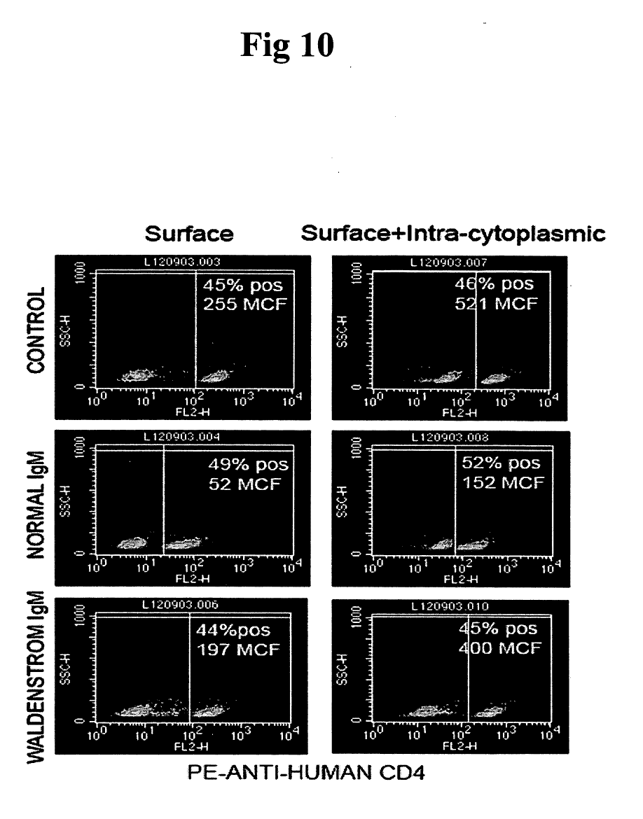

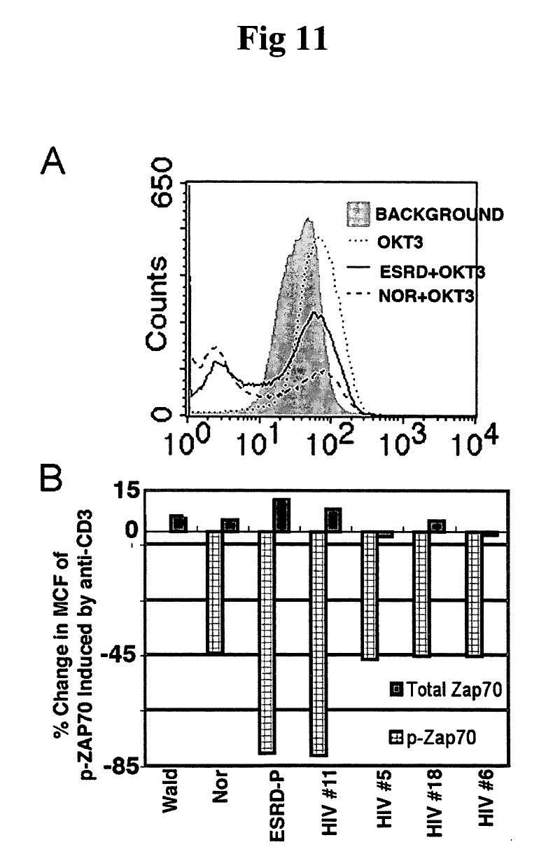

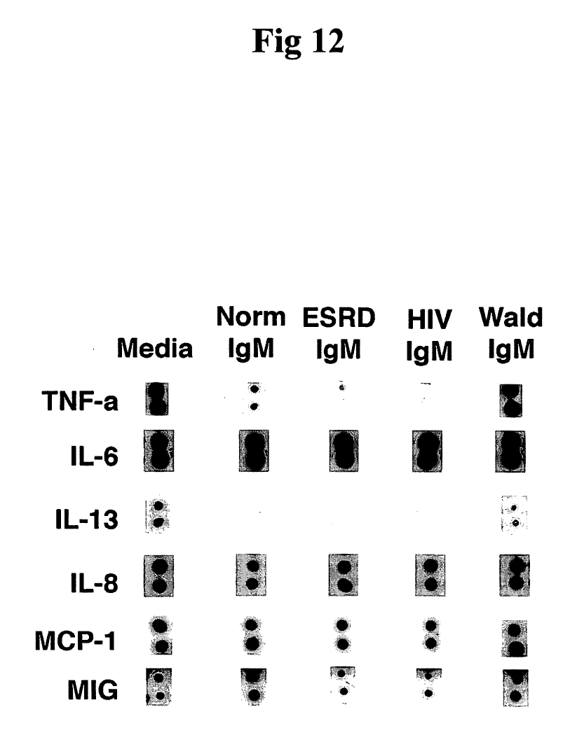

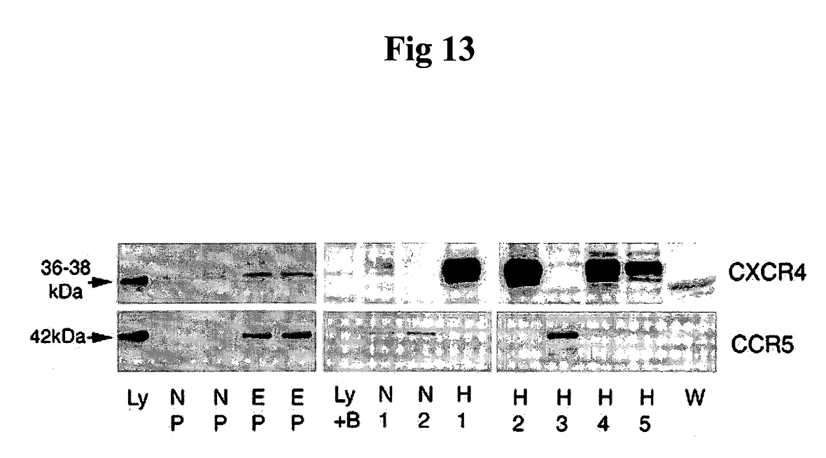

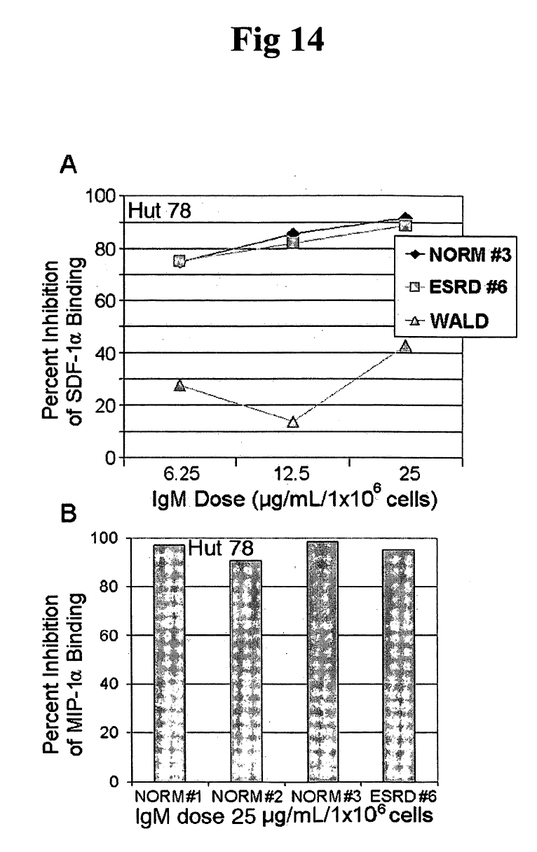

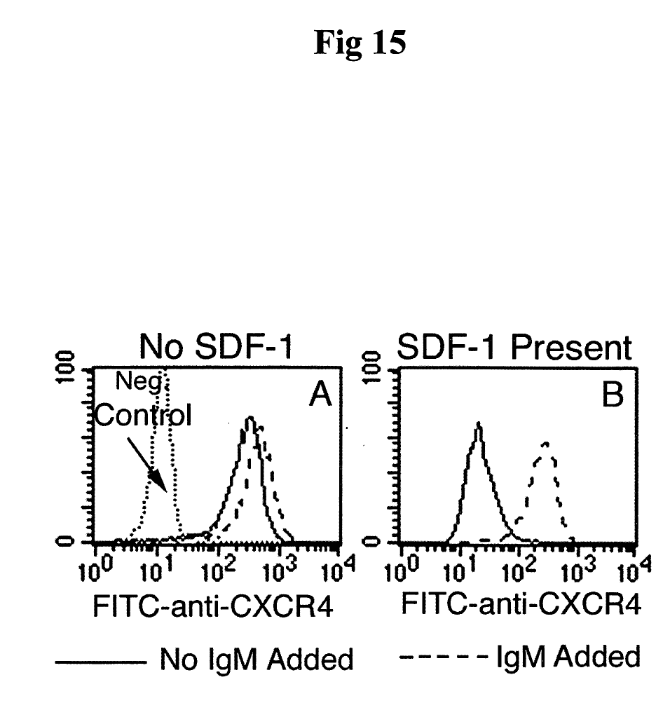

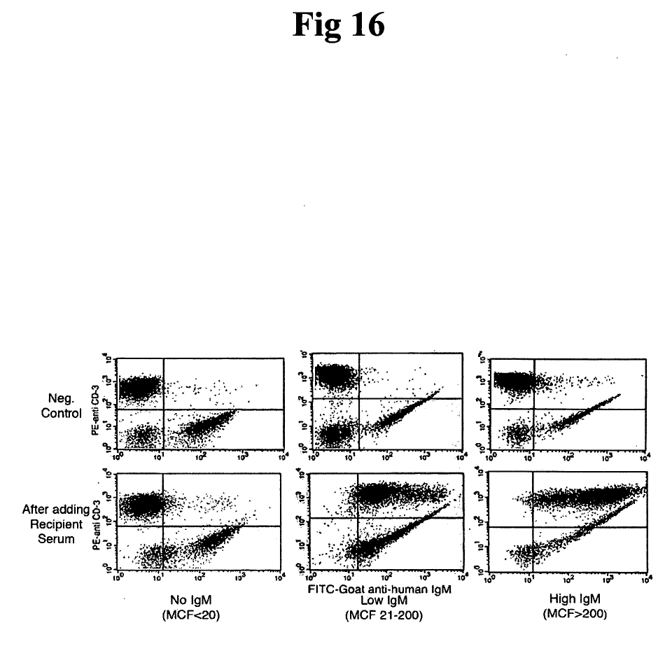

### MODES FOR CARRYING OUT INVENTION

While not wishing to be bound to any particular theory, there are several possible explanations for the entry of the HIV-1 virus into cells and increased viral replication despite the presence of a good level of IgM autoantibody to chemokine receptor during the asymptomatic state. One such explanation is the possibility that there exists a delicate balance between these low-affinity binding IgM antibodies and the viral load. Factors that predispose an individual to an increased viral load or that inhibit the B cells secreting IgM autoantibodies will lead to viral entry into cells and to disease progression. It is also possible that the recently described subset of B cells expressing CD4, CXCR4 and CCR5 receptors may be the same subset that secretes IgM autoantibodies. Over several months or years, this B cell subset could be exhausted or could be infected with HIV-1, thereby leading to a decrease in antibody production. Additionally, one cannot underscore the importance of other host factors (e.g., anti-viral IgG antibodies, chemokines and complement and cytotoxic T cells) that decrease the viral load. Perturbation in any of these host defense mechanisms could lead to an increased viral load.

Secondly, it is possible that in some HIV-1 infected individuals, IgM anti-lymphocyte antibody may only partially prevent entry of certain HIV-1 viral isolates, as indicated by some of the studies herein. This latter mechanism may provide another explanation for disease progression despite the presence of IgM anti-chemokine receptor autoantibodies.

That IgM autoantibodies inhibit HIV-1 virus from cell entry and replication supports the premise for a protective role mediated by these IgM anti-leukocyte antibodies. The use of isolated human IgM anti-leukocyte antibodies to reduce HIV-1 infectivity (i.e., through receptor blockade and/or inactivation of cells) is an alternative approach for passive immunization. Receptor blockade by administering to an individual, IgM with reactivity to a broad range of chemokine and other receptors present on the lymphocytes may be particularly useful in situations where the HIV-1 virus switches its receptor usage, e.g., from CCR5 to CXCR4. Maintaining increased levels of such protective antibodies could also increase the latency period after HIV-1 infection. Additionally, it may be possible to design immunization strategies or vaccines that enhance in-vivo IgM anti-lymphocyte NAA that are inhibitory to HIV-1 infectivity.

Diseases associated with tissue-specific inflammatory processes, angiogenesis and growth (and spread) of malignant cells are controlled by chemokines, cytokines, chemokine receptors and other receptors that activate (or inhibit) cell function. Such receptors are present on all leucocytes, endothelial cells and malignant cells. IgM anti-lymphocyte NAA, by binding to chemokine and other receptors (e.g. lipid rafts, CD4 and CD3) could provide a regulatory role in the above-mentioned disorders or processes. The use of isolated IgM, especially IgM antibodies that inhibit chemokine receptor function or inhibit cell activation (i.e. with potential of causing apoptosis of malignant cells) or inhibit chemokine and cytokine production, would be particularly beneficial for inflammatory processes or growth and spread of malignant cells. Studies in renal transplant recipients clearly indicate that chemokines and chemokine receptors have a role in the rejection process. Data in this regard is reviewed in Hancock, W. W, J of Am Soc Nephrol 13: 821-824, 2002 and the material in this reference is incorporated herein by reference. Hence, the finding that kidney transplant recipients, with low or high levels of IgM anti lymphocyte antibodies, have no or minimal acute rejections would support the concept that IgM anti-lymphocyte antibodies inhibit chemokine receptor function and lymphocyte activation. One could employ passive immunization technique or alternatively design immunization strategies that specifically enhance in-vivo production of IgM anti-lymphocyte NAA (with inhibitory effect on chemokine receptor function or cell activation) to treat the various inflammatory processes and growth (and spread) of malignant cells. Inhibition of TNF-α is particularly important as prior art has shown that inhibitors of TNF-α (e.g. antibodies to TNF-α) can suppress inflammation in patients with rheumatoid arthritis and Crohn's disease (see Feldmann M, Annual Rev Immunol 2001, vol 19, p163-196, and Sandborn W J Inflamm Bowel Dis. 1999, vol 1 p 119-133 and the material in these references is incorporated herein by reference).

The source of IgM antibodies may be heterologous, autologous or allogeneic. IgM antibodies with specificity for chemokine and other receptors on the leukocyte may be raised in vivo (i.e., in mice or other animals or in humans) or in vitro using cell culture techniques.

For example, IgM antibodies may be produced either in vivo or in vitro by genetic engineering whereby genes specific for IgM anti-lymphocyte antibodies are introduced into antibody-producing cells. These antibody-producing cells may then be introduced into an infected human or into immunodeficient animals where the cells produce IgM antibodies. In the alternative, these antibody-producing cells may be grown in vitro using hybridoma or other technology.

IgM antibodies with specificity for chemokine receptors or non-chemokine receptors may also be produced by isolating human or animal antibody-producing cells specific for IgM anti-lymphocyte antibodies and enhancing antibody production by such cells using hybridoma or other technology, including introduction of the cells into animals or humans. For example, human lymphocytes may be transplanted into immunodeficient mice, and the lymphocytes may then be stimulated with an agent that will activate B cells such as lipopolysaccharide (“LPS”)

Another method of producing IgM antibodies is by isolating human antibody-producing cells capable of generating human IgM from animals such as, for example, the XenoMouse™. IgM antibody production by such cells may then be enhanced in vitro employing hybridoma or other technology such as, for example, stimulating the isolated lymphocytes with LPS or other agent that will activate the cells, e.g., the EBV virus.

IgM antibodies may also be produced in vitro by isolating, from an individual, lymphocytes that can be then transformed with the EBV virus and introduced in a culture. A subset of these EBV transformed B lymphocytes will secrete IgM antibodies such that the resulting culture fluid contains these antibodies. These EBV transformed B lymphocytes, secreting IgM can then be fused with a non-secreting myeloma cell line to develop hybridomas.

In addition, viruses, bacteria and other antigens (e.g., mitogens) may be used to stimulate B cells in vivo to generate IgM antibodies to leukocytes.

IgM antibodies produced outside an infected individual may be delivered to the individual by one of several routes of administration including, but not limited to, intravenous, intraperitoneal, oral, subcutaneous, and intramuscular delivery.

IgG, IgD, IgE and IgA isotypes of naturally occurring autoantibodies (i.e. NAA) have also been described in prior art. The present invention also relates to IgG, IgD, IgE and IgA isotypes especially since there is prior art describing technology for the molecular cloning of antibodies virus using combinatorial phage display libraries containing genes coding for antibody fragment of the IgM, IgD, IgA or IgG phenotype as well as genes for the naturally expressed human antibody repertoire. (See Raum T, Cancer Immunology, Immunotherapy 2001, vol. 50, p. 141-50, Burioni R, Research in Virology 1998, vol. 149, p. 321-25 and the material in these references is incorporated herein by reference). Human IgM natural antibodies against a lymphocyte receptor, can through this technology, be switched to another antibody phenotype. All antibody isotypes in this invention includes intact immunoglobulins or fragments of these antibodies. As such, throughout the specification and claims the use of the term “antibodies” or auto antibodies” includes naturally occurring antibodies of all isotypes used as intact immunoglobulins or fragments of these antibodies.

Having now fully described the invention with reference to certain representative embodiments and details, it will be apparent to one of ordinary skill in the art that changes and modifications can be made thereto without departing from the spirit or scope of the invention as set forth herein.

The material in the 26 references listed below is herein incorporated in this application to provide more detailed information that will enable the claims.

References

1. Banapour B., Sematinger J., and Levy J. B. (1986) The AIDS-associated retrovirus is not sensitive to lysis or inactivation by human serum. Virology, 152, 268-271.
2. Berberian L., Goodglick K., Kipps T. J., Braun J. (1993) Immunoglobulin VH3 gene products: natural ligands for HIV gpl2O. Science 261 Sep 17; 1588-91.
3. Cervenak J., Kiss K., & Uher F. (1999) Partial characterization of two lymphocyte specific natural autoantibodies isolated from new born mice. Acta Microbiologica et Immunologica Hungarica, 53-62.
4. Frenkel, N., Schirmer, E. C., Katsafanas, G., & June, C. H. (1990) T-cell activation is required for efficient replication of human herpesvirus 6. J Virol., 64, 4598-4602.
5. Gerard, C. & Rollins, B. J. (2001) Chemokines and disease. Nat.Immunol., 2, 108-115.
6. Griggi T., Bauer R., Garofalo T., Kukel S., Lenti L., Massetti A. P., Muller C., Sorice M., & Pointieri G. M. (1994) Autoantibodies against ganglioside GM3 represents a portion of antilymphocytic antibodies in AIDS patients. Scan J of Immunol, 40, 77-82.
7. Hancock, W. W. (2002) Chemokines and transplant immunobiology. J Am. Soc. Nephrol, 13, 821-824.
8. Hansson, G. K., Libby, P., Schonbeck, U., & Yan, Z. Q. (2002) Innate and adaptive immunity in the pathogenesis of atherosclerosis. Circ. Res., 91, 281-291.
9. Hoshino H., Tanaka H., Miwa M., Okada H., 1984 Human T-cell leukaemia virus is not lysed by human serum. Nature, 310, 324-325.
10. Huber, B. T., Hsu, P. N., & Sutkowski, N. (1996) Virus-encoded superantigens. Microbiol. Rev., 60, 473482.
11. Jenkins, M. K., Khoruts, A., Ingulli, E., Mueller, D. L., McSorley, S. J., Reinhardt, R. L., Itano, A., & Pape, K. A. (2001) In vivo activation of antigen-specific CD4 T cells. Annu. Rev. Immunol., 19, 23-45.
12. Lacroix-Desmazes S., Kaveri S. V., Mouthon L., Ayouba A., Melanchere E., Coutinho A., & Kazatchkine M. D. (1998) Self-reactive antibodies (natural autoantibodies) in healthy individuals. J of Immunol Methods, 216, 117-137.
13. Llorente M. Sanchez-Palomion S, Manes S, Lucas, P, Kremer K, De Alboran I. M., Joran J. L., Alcami J, Del Real G, Martizne-A. C. (1999) Natural human antibodies retrieved by phage display libraries from healthy donors: polyreactiveity and recognition of human immunodeficiency virus type 1 gp 120 epitopes. Scand J Immunol., 50, 270-279.
14. Lobo P. I., Westervelt F. B., Rudolf L, & White C (1980) Cold lymphocytotoxins-an important cause of acute tubular necrosis (ATN) occuring immediately post-transplant. Lancet, 2, 879-882.
15. Muller, A., Homey, B., Soto, H., Ge, N., Catron, D., Buchanan, M. E., McClanahan, T., Murphy, E., Yuan, W., Wagner, S. N., Barrera, J. L., Mohar, A., Verastegui, E., & Zlotnik, A. (2001) Involvement of chemokine receptors in breast cancer metastasis. Nature, 410, 50-56.
16. Nakamura, M., Burastero, S. E., Ueki, Y., Larrick, J. W., Notkins, A. L., & Casali, P. (1988) Probing the normal and autoimmune B cell repertoire with Epstein-Barr virus. Frequency of B cells producing monoreactive high affinity autoantibodies in patients with Hashimoto's disease and systemic lupus erythematosus. J Immunol., 141, 4165-4172.
17. Olson, T. S. & Ley, K. (2002) Chemokines and chemokine receptors in leukocyte trafficking. Am. J Physiol Regul. Integr. Comp Physiol, 283, R7-28.
18. Onuffer, J. J. & Horuk, R. (2002) Chemokines, chemokine receptors and small-molecule antagonists: recent developments. Trends Pharmacol. Sci., 23, 459-467.
19. Perkins, D. L. (1998) T-cell activation in autoimmune and inflammatory diseases. Curr. Opin. Nephrol. Hypertens., 7, 297-303.
20. Pullar C. E, Morris P. J, Wood K. J (2003) Altered proximal T-cell receptor signaling evens in mouse CD4+T cells in the presence of anti-CD4 monoclonal antibodies: evidence for reduced phosphorylation of Zap-70 and LAT. Scand J Immunol., 57, 333-341.
21. Rodman, T. C., Pruslin F. H., To S. E., and Winston R., (1992) Human immunodeficiency virus (HIV) Tat-reactive antibodies present in normal HIV- negative sera and depleted in HIV-positive sera. Identification of the epitope J Exp. Med., 175: 1247-1253.
22. Stein, B. S. & Engleman, E. G. (1991) Mechanism of HIV-1 entry into CD4+T cells. Adv.Exp.Med.Biol., 300, 71-86.
23. Stimmler, M. M., Quismorio, F. P. J., McGehee, W. G., Boylen, T., & Sharma, O. P. (1989) Anticardiolipin antibodies in acquired immunodeficiency syndrome. [see comments]. Archives ofinternal Medicine, 149, 1833-1835.
24. Sutkowski, N., Conrad, B., Thorley-Lawson, D. A., & Huber, B. T. (2001) Epstein-Barr virus transactivates the human endogenous retrovirus HERV-K18 that encodes a superantigen. Immunity., 15, 579-589.
25. Torbett, B., Picchio G. R., & Mosier D. E. (1991) hu-PBL-SCID mice: a model for human immune function, AIDS and lymphomagenesis. Immunol Reviews, 124, 139-164.
26. Werlen, G. & Palmer, E. (2002) The T-cell receptor signalosome: a dynamic structure with expanding complexity. Curr. Opin. Immunol., 14, 299-305.

1. A method of treating human diseases or disorders, comprising administering to the individual isolated naturally occurring antibodies (NAAs) or fragments thereof or cells producing NAA or enhancing in-vivo production of NAA having binding specificity to cell surface receptors present on lymphocytes.

2. The method of claim 1, wherein the receptors are chemokine receptors.

3. The method of claim 2, wherein, the chemokine receptors are selected from the group consisting of CCR5, CXCR4, CCR2b, CCR3 and other chemokine receptors that have reactivity to naturally occurring antibodies.

4. The method of claim 1, wherein, the cell surface receptors present on lymphocytes, are non-chemokine receptors.

5. The method of claim 4, wherein, the cell surface receptor is selected from the group consisting of CD3, CD4, CD2, CD80, CD86 receptor, lipid raft, and other non-chemokine receptors on the cell membranes.

6. The method of claim 1, wherein anti-lymphocyte NAA bind to chemokine and non-chemokine receptors present on lymphocytes and wherein these anti-lymphocyte NAA are polyreactive and wherein anti-lymphocyte NAA bind to similar chemokine and non-chemokine receptors present on non-lymphocyte leucocytes, or endothelial cells, or malignant cells.

7. The method of claim 1, wherein, the anti-lymphocyte NAA having specificity to cell surface receptors present on leucocytes, endothelial cells, and malignant cells, are selected from the group consisting of human, and animal, IgM NAA.

8. The method of claim 7, wherein, the anti-lymphocyte NAA can be selected from monoclonal NAA or polyclonal NAA, or synthetic NAA, or recombinant NAA or antibody fragments of NAA, or NAA of all isotypes generated from combinatorial libraries containing naturally expressed Ig repertoires.

9. The method of claim 1, wherein, the human disease or disorder, comprises virus mediated disease, autoimnune disease, inflammatory states, and cellular malignancies.

10. The method of claim 6, wherein NAA can inhibit activation of T cells or other cells.

11. The method of claim 6, wherein NAA can inhibit chemotaxis, and chemokinesis of cells.

12. The method of claim 6, wherein NAA can inhibit chemokine and cytokine production.

13. The method of claim 12, wherein the chemokine and cytokine is selected form the group consisting of TNF-α, IL-13, MDP, TARC, and other chemokine or cytokine.

14. The method of claim 6, wherein anti-lymphocyte NAA can enhance death of cells.

15. The method of claims 2, 4, 6, 7, 9, 10, or 12 wherein the viral mediated disease caused by HIV-1, or other viruses infecting lymphocytes or other cells, and wherein these viruses use for cell entry chemokine or non-chemokine receptors present on lymphocytes or other cells, and wherein virus cell entry and/or replication is enhanced by activation of T cells or other cells, and wherein viral cell entry and/or replication is inhibited by NAA that inhibits viral entry, activation and/or proliferation of T cells and other cells.

16. The method of claims 2, 4, 6, 7, 9 10, 11 or 12, wherein the autoimmune disease is selected from the group of systemic lupus erythematosus, rheumatoid arthritis, Type 1 diabetes mellitus, multiple sclerosis, vasculitis, and other autoimmune conditions, in which the autoimmune inflammatory process is enhanced by or mediated by T cell activation, and chemokine receptors, chemokines and cytokines, and wherein NAA with binding specificity to chemokine and non-chemokine receptors will inhibit the autoimmune inflammatory process.

17. The method of claims 2, 4, 6, 7, 9, 10, 11 or 12, wherein the inflammatory state is selected from the group of asthma, sarcoidosis, atherogenesis and atherosclerosis, or allograft and xenograft rejections, in which the inflammatory process is enhanced or mediated by T cell activation, and chemokine receptors, chemokines and cytokines, and wherein NAA with binding specificity to chemokine and non-chemokine receptors will inhibit the inflammatory process.

18. The method of claims 2, 4, 6, 7, 9, 10, 11, 12 or 14, wherein the cellular malignancy involves lymphoid or non-lymphoid malignancies, and wherein NAA bind to chemokine receptors and non-chemokine receptors on lymphocytes, and other cells, and wherein NAA inhibits activation of cells, inhibits cell proliferation and enhances apoptosis of tumor cells.

19. The method of claim 1, wherein therapy would comprise administering isolated NAA to an individual to inhibit progression of disease processes or prevent disease processes.

20. The method of claim 15, wherein NAA binds to cell surface receptors important in inhibiting activation of T cells, and other cells, and wherein NAA inhibit viral infectivity of cells, and wherein such viruses include HIV-1, EBV, CMV, Rabies virus, Polio virus, Herpes virus 6, influenza virus, and Ebola virus.

21. The method of claim 4 wherein NAA binds to non-chemokine cell surface receptors present on lymphocytes and other cells and wherein viruses use said receptors for viral entry and wherein such viruses include HIV-1, EBV, CMV, Rabies virus, Polio virus, Herpes virus 6, influenza virus, and Ebola virus and wherein NAA inhibit entry of these viruses through non-chemokine receptors.

22. The method of claim 1, wherein the isolated anti-lymphocyte NAA are administered to the individual by oral routes, by subcutaneous routes, intravenously, intraperitoneally, or intramuscularly or their production is enhanced in-vivo with one or more agents elected from the group consisting of viruses, inactive bacteria, antigens, and mitogens.

23. The method of claim 1, wherein animal or human anti-leucocyte NAA are produced to treat human diseases or disorders, comprising introducing genes specific for anti-leucocyte NAA into antibody-producing cells, and producing the anti-leucocyte NAA antibodies in vitro or in vivo.

24. The method of claim 1, wherein animal or human anti-leucocyte NAA are produced to treat human diseases or disorders, comprising isolating human, or animal antibody producing cells and enhancing production of NAA in-vitro or in-vivo by the antibody producing cells.

25. The method of claim 1, wherein anti-leucocyte NAA production comprises isolating human antibody-producing cells from animals capable of generating human NAA and enhancing production of anti-leucocyte NAA in vitro or in vivo by the antibody-producing cells.

26. The method of claim 1, where anti-leucocyte NAA are produced from combinatorial libraries that include naturally expressed Ig repertories.

27. The method of claim 1, wherein anti-leucocyte NAA are produced in-vitro using viruses, bacteria, antigens, alloantigens or autoantigens either singly or in different combinations.

28. The method of claim 1, wherein anti-leucocyte NAA are produced in vivo by injecting one or more individuals or animals with one or more elected from the group consisting of viruses, inactive bacteria, viral and bacterial products, fungal products, plant antigens, mitogens, alloantigens or autoantigens either singly or in different combinations.

29. The method of claim 1, wherein the anti-lymphocyte NAA comprise antibodies of all immunoglobulin isotypes or classes.

30. The method of claim 1, wherein isolated anti-lymphocyte NAA comprise antibodies isolated from humans or animals or antibodies isolated after in-vitro production.
